SEQUENCE LISTING (12 sequences)

SEQ ID NO: 1 - DNA, 25 bp, Artificial Sequence
  Description of Artificial Sequence 35S CaMV promoter
cgaaggatagtgggattgtgcgtca

SEQ ID NO: 2 - DNA, 25 bp, Artificial Sequence
  Description of Artificial Sequence 35S CaMV promoter
aaggtggctcctacaaatgccatca

SEQ ID NO: 3 - DNA, 27 bp, Artificial Sequence
  Description of Artificial Sequence soybean lectin gene
caacgaaaacgagtctggtgatcaagt

SEQ ID NO: 4 - DNA, 27 bp, Artificial Sequence
  Description of Artificial Sequence soybean lectin
tggtggaggcatcataggtaatgagaa

SEQ ID NO: 5 - DNA, 26 bp, Artificial Sequence
  Description of Artificial Sequence E. coli 0157H7 DNA
atgcagacccgctggagtttgagaaa

SEQ ID NO: 6 - DNA, 26 bp, Artificial Sequence
  Description of Artificial Sequence E. coli 0157H7 DNA
tacctgaggcagtagcgataatgagc

SEQ ID NO: 7 - DNA, 32 bp, Artificial Sequence
  Description of Artificial Sequence SV-40 DNA
ttaaaaagctaaaggtacacaatttttgagca

SEQ ID NO: 8 - DNA, 32 bp, Artificial Sequence
  Description of Artificial Sequence SV-40 DNA
aaaagctgcactgctatacaagaaaattatgg

SEQ ID NO: 9 - DNA, 206 bp, Artificial Sequence
  Description of Artificial Sequence 35S CaMV
aaggtggctcctacaaatgccatcattgcgataaaggaaaggctatcgttcaaaatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcg

SEQ ID NO: 10 - DNA, 185 bp, Artificial Sequence
  Description of Artificial Sequence soybean lectin gene Le1
caacgaaaacgagtctggtgatcaagtcgtcgctgttgagtttgacactttccggaactcttgggatccaccaaatccacacatcggaattaacgtcaattctatcagatccatcaaaacgacgtcttgggatttggccaacaataaagtagccaaggttctcattacctatgatgcctccacca

SEQ ID NO: 11 - DNA, 527 bp, Artificial Sequence
  Description of Artificial Sequence E. coli 0157H7 DNA
atgcagacccgctggagtttgagaaaaagtacggctctcagattgagttaatttttcgttttcttgatcacgcctttgcgactggcgtgctcgggtaaaagaggtgactgatgctcatagatttggttttaccttacccgccgacggtgaacacctactggcgacgtcgtggcagcacatattttgtatcaaaagccggtgagcgttatcgccgggctgtggcgcttattgttcgccagcagcggctgaaattaagcctgtccggaaggctggcgatgaagattattgccgagccaccggataagcgccgccgtgacctggacaatgttctgaaagcgccgctggatgcgctgacgcatgcggggttgctaatggacgatgagcagtttgatgaaatcaatattgtgcgcggtcagctcgttcctggtgagcggctggggataaaaatcacagaactggagtgcgcatgaataaccactatttacagtttgtgcgtgagctgctcattatcgctactgcctcaggta

SEQ ID NO: 12 - DNA, 167 bp, Artificial Sequence
  Description of Artificial Sequence SV-40 Large-T antigen DNA
ttaaaaagctaaaggtacacaatttttgagcatagttattaatagcagacactctatgcctgtgtggagtaagaaaaaacagtatgttatgattataactgttatgcctacttataaaggttacagaatatttttccataattttcttgtatagcagtgcagctttt

(19) United States
(12) Patent Application Publication
(10) Pub. No.: US 2004/0053230 A1
(43) Pub. Date: Mar. 18, 2004

(54) Real time quantitative pcr with intercalating dye for single and multiplex target dna

(76) Inventors: Michael Edward Schaffer, Boothwyn, PA (US); Susan Yen-Tee Tseng, Hockessin, DE (US)

Correspondence Address: Kening Li, E I du Pont de Nemours & Company, Legal Patents, Wilmington, DE, 19898, US

(21) Appl. No.: 10/204,889

(22) PCT Filed: Mar. 7, 2001
(86) PCT No.: PCT/US01/07101

(51) Int. Cl. C12Q 1/68; G06F 19/00; G01N 33/48; G01N 33/50

(52) U.S. Cl. 435/6; 702/20

(57) ABSTRACT

The PCR-based, dsDNA quantification method monitors the fluorescence of a target, whose melting characteristics is predetermined, during each amplification cycle at selected time-points. Fluorescence is measured immediately after the annealing phase (F_{E} at T_{E}), immediately below (F_{MS} at T_{MS}) and above (F_{ME} at T_{ME}) the melting of the target/amplicon. A change in slope from a baseline slope (S_{B}=−(F_{MS}−F_{E})/(T_{MS}−T_{E})) to a melting phase slope (S_{M}=−(F_{ME}−F_{MS})/(T_{ME}−T_{MS}) indicates a specific amplification. The number of amplification cycles (C_{T}) it takes for the quantity (S_{M}−S_{B}) to become greater than zero correlates with the starting concentration of the target (C). The concentration of the target in a sample is determined by comparing the value of C_{T} for the sample with a standard curve. By selecting targets with distinguishable melting curve characteristics, multiple targets can be simultaneously detected.

This application claims the benefit of U.S. Provisional Application No. 60/187537, filed Mar. 7, 2000.

### FIELD OF THE INVENTION

This invention relates to the field of molecular biology and more particularly to DNA-based diagnostic protocols.

### BACKGROUND OF THE INVENTION

In many instances it is desirable to quantify the level of a target nucleic acid in a sample. For example, it may be important to determine the amount of a pathogenic organism in a food or water sample, or the amount of a genetically modified organism (GMO) in a crop.

The polymerase chain reaction (PCR) is a highly sensitive-and powerful method for such detections. Many specific adaptations of the PCR technique are known in the art for both qualitative and quantitative detections. In particular, methods are known to utilize fluorescent dyes for detecting and quantifying amplified PCR products. In situ amplification and detection, also known as homogenous PCR, have also been previously described. See e.g. Higuchi et al., (Kinetics PCR Analysis: Real-time Monitoring of DNA Amplification Reactions, Bio/Technology, Vol 11, pp 1026-1030 (1993)), Ishiguro et al., (Homogeneous quantitative Assay of Hepatitis C Virus RNA by Polymerase Chain Reaction in the Presence of a Fluorescent Intercalater, Anal. Biochemistry 229, pp 20-213 (1995)), and Wittwer et al., (Continuous Fluorescence Monitoring of Rapid cycle DNA Amplification, Biotechniques, vol.22, pp 130-138 (1997.))

In these methods a fluorescence signal is acquired once per cycle during the annealing/elongation phase of the PCR reaction. The fluorescence so measured, however, represents the total fluorescence of the mixture of specifically amplified target PCR products (target amplicon), as well as non-specific amplicons, which include single primer products, primer-dimers, and other aberrant amplicons. These previously disclosed methods cannot differentiate between specific and non-specific amplicons, and are particularly problematic when the target copy numbers are low.

In addition, these methods can only be used to detect a single target DNA in each PCR reaction, but often it is desired to simultaneously detect multiple target nucleic acids in one PCR reaction.

Furthermore, there is a need to have a positive control in the same reaction wherein the sample DNA is simultaneously amplified. Such an “internal positive control” would serve as both as a positive control for the PCR reaction, and to calibrate PCR reactions whose amplification efficiency varies due to impurities introduced by the test sample.

There is also a need to have a quantitative PCR method that reliably determines target nucleic acid concentrations in a sample at low copy number, that can distinguish target amplicons and non-specific amplicons, and that can be used to detect and quantify multiple target nucleic acids simultaneously. The inventions disclosed herein fulfill these and other needs.

### SUMMARY OF THE INVENTION

A method for detecting in real time the amount of a target nucleic acid molecule in a sample, wherein the melting of the target nucleic acid molecule starts at a temperature T_{MS} and completes at a temperature T_{ME}, the method comprising:

A. Establishing a standard curve by: i) PCR-amplifying, in the presence of a suitable fluorescent dye, the target nucleic acid molecule, with a known starting concentration (C) through cycles of denaturing, annealing, and chain extension, wherein the fluorescence is increased when the dye is combined with a double-stranded nucleic acid molecule, wherein the chain extension occurs at a chain extension temperature T_{E}; ii) measuring the fluorescence (F) during each amplification cycle at the temperature immediately before the temperature starts to increase from T_{E} (F_{E} at T_{E}), at any temperature point (T_{B}) in between T_{E} and T_{MS} (F_{B} at T_{B}), at T_{MS} (F_{MS} at T_{MS})and at T_{ME} (F_{ME} at T_{ME}); iii) calculating a baseline slope (S_{B}), defined by negative (F_{B} minus F_{E}), divided by (T_{B} minus T_{E}), and an amplicon melting phase slope (S_{M}), defined by negative (F_{ME} minus F_{MS}) divided by (T_{ME} minus T_{MS}); iv) recording the number of PCR cycles (N) required for the quantity (S_{M} minus S_{B}) to first become greater than zero; v) repeating steps i) through v) for a suitable range of concentrations of interest; and vi) plotting C against C_{T} to obtain a standard curve for the target nucleic acid sequence; and

B. Repeating steps (A) (i) through (A)(v) for a sample containing an unknown concentration of the target nucleic acid molecule, to obtain an C_{T} value for the sample, and determining the target nucleic acid molecule concentration via the standard curve.

The inventive method described above may also be used to detect multiple targets in a sample. Specifically, when the sample contains n target nucleic acid molecules, wherein n is an integer greater than one, wherein the melting of the first target nucleic acid molecules starts at a temperature T_{MS1} and completes at a temperature T_{ME1}, the melting of the second target nucleic acid molecule starts at a temperature T_{MS2} and completes at a temperature T_{ME2}, the melting of the (n−1)^{th} target nucleic acid molecule starts at a temperature T_{MS(n-1)} and completes at a temperature T_{ME(n-1)}, the melting of the n^{th} target nucleic acid molecule starts at a temperature T_{MSn} and completes at a temperature T_{MEn}, and wherein T_{MSn} is greater than T_{ME(n-1)}. The method for multiplex detection comprises:

A. Establishing a standard curve for each of the target nucleic acid molecule according to the method of for single target detection;

B. Simultaneously PCR amplifying a sample containing an unknown concentration of the target nucleic acid molecules, to obtain an N₁, N₂ . . . and Nₙ value for the sample, and determining the target nucleic acid molecule concentrations via the standard curve.

According to a preferred embodiment, the first and the second target nucleic acid molecules reside on the same genome of an organisms, and the copy number per genome for the first target nucleic acid molecule is known, whereby the copy number per genome for the second target nucleic acid molecule is determined.

According to another embodiment of the invention, the first target nucleic acid is an invertase gene or a lectin gene, and wherein the second target nucleic acid is selected from the group consisting of the 35 S promoter of CaMV, a Cry9C gene, and an GA21 gene.

According to a particularly preferred embodiment, the target nucleic acid molecule is selected from the group consisting of SEQ ID NO: 11, SEQ ID NO: 12, SEQ ID NO: 9, and SEQ ID NO: 10.

According to a most preferred embodiment, the target nucleic acid molecule is a nucleic acid fragment is part of a transgene contained in a genetically modified organism.

### BRIEF DESCRIPTION OF THE DRAWINGS

FIG. 1: Melting profile of a fragment of the CaMV 35 S promoter, showing the relationship between temperature and relative fluorescence intensity, and the baseline slopes (S_{B}) and the amplicon slope (S_{M}).

FIG. 2: Real-time quantitative PCR amplification of the amplicon in FIG. 1, showing the relationship between template starting concentration and the delta slope difference (S_{M}-S_{B}).

FIG. 3: Standard Curve for the amplicon of FIG. 1, showing the relationship between the log of initial target concentrations and threshold cycle number in PCR.

FIG. 4: Melting profile of the lectin Amplicon, showing the relationship between temperature and relative fluorescence intensity, and the baseline slopes (S_{B}) and the amplicon slope (S_{M}).

FIG. 5: Standard Curve for the amplicon of FIG. 3, showing the relationship between the log of initial target concentrations and threshold cycle number in PCR.

FIG. 6: Melting profile of E. coli O17:H7 Amplicon, showing the relationship between temperature and relative fluorescence intensity, and the baseline slopes (S_{B}) and the amplicon slope (S_{M}).

FIG. 7: Real-time quantitative PCR amplification of the amplicon in FIG. 6, showing the relationship between template starting concentration and the delta slope difference (S_{M}-S_{B}).

FIG. 8: Standard Curve for the amplicon of FIG. 6, showing the relationship between the log of initial target concentrations and threshold cycle number in PCR.

FIG. 9: Melting profile of the SV40 amplicon, showing the relationship between the temperature and relative fluorescence intensity, and the baseline slopes (S_{B}) and the amplicon slope (S_{M}).

FIG. 10: Standard Curve for the amplicon of FIG. 9, showing the relationship between the log of initial target concentrations and threshold cycle number in PCR.

FIG. 11: Melting profile of a mixture of both the E. coli O157:H7 and the SV40 amplicons. Mixture and slope determination.

FIG. 12: Standard Curve for the SV40 amplicon from a multiplex Q-PCR assay.

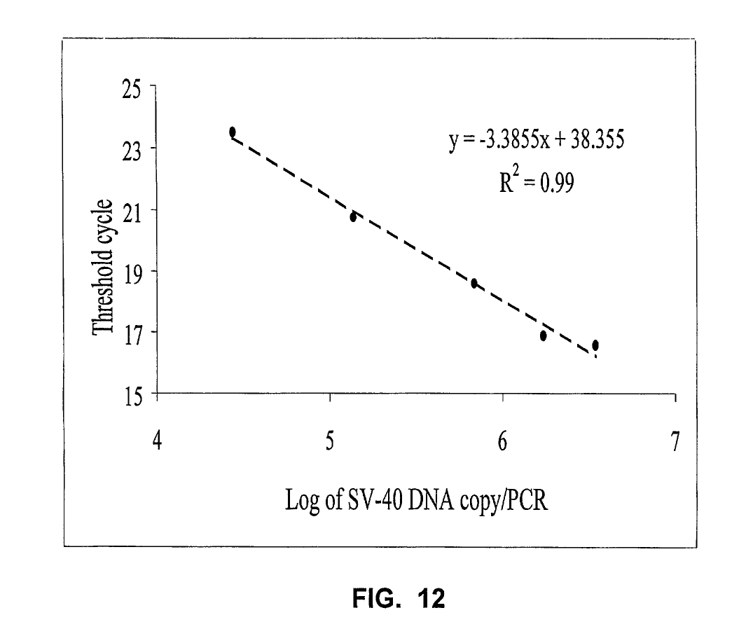

FIG. 13: Standard Curve for the E. coli O157:H7 amplicon from a multiplex Q-PCR Assay.

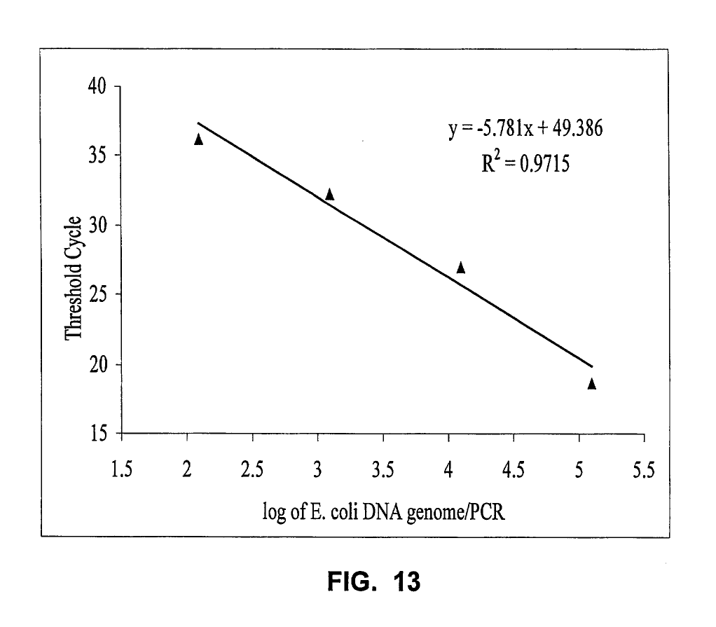

FIG. 14: 35S CaMV melting profile and signal determination. This Figure shows the relationship between temperature and relative fluorescence intensity of a specific CaMV amplified product, and also demonstrates the difference between amplicon signal and background fluorescence.

FIG. 15: Real-time Quantitative PCR reaction of CaMV. This figure demonstrates the use of an average base line to set threshold, and shows the relationship between the number of thermal cycle of each concentration of CaMV samples and the fluorescence signal difference (specific−background).

FIG. 16: Determination of threshold cycle for an amplicon. The cycles used to define the initial fluorescence (F_{I}) and the background threshold fluorescence (F_{B}) are shown. The threshold cycle (C_{T}) can be determined as a function of the amplicon production and F_{B}.

### BRIEF DESCRIPTION OF THE SEQUENCE LISTING

SEQ ID NO: 1 is the sequence of a synthetic oligonucleotide encoding a portion of the promoter region for the cauliflower mosaic virus 35S promoter (35S CaMV). When used in a PCR reaction with the oligonucleotide represented in SEQ ID NO: 2 the fragment represented in SEQ ID NO: 9 is produced.

SEQ ID NO: 2 is the sequence of a synthetic oligonucleotide encoding a portion the promoter region for the cauliflower mosaic virus 35S promoter (35S CaMV). When used in a PCR reaction with the oligonucleotide represented in SEQ ID NO: 1 the fragment represented in SEQ ID NO: 9 is produced.

SEQ ID NO: 3 is the sequence of a synthetic oligonucleotide encoding a portion of the soybean lectin gene Le-1. When used in a PCR reaction with the oligonucleotide represented in SEQ ID NO: 4 the fragment represented in SEQ ID NO: 10 is produced.

SEQ ID NO: 4 is the sequence of a synthetic oligonucleotide encoding a portion of the soybean lectin gene Le-1. When used in a PCR reaction with the oligonucleotide represented in SEQ ID NO: 3 the fragment represented in SEQ ID NO: 10 is produced.

SEQ ID NO: 5 is the sequence of a synthetic oligonucleotide encoding a portion of the genome unique to Escherichia coli 0157:H7. When used in a PCR reaction with the oligonucleotide represented in SEQ ID NO: 6 the fragment represented in SEQ ID NO: 11 is produced.

SEQ ID NO: 6 is the sequence of a synthetic oligonucleotide encoding a portion of the genome unique to Escherichia coli 0157:H7. When used in a PCR reaction with the oligonucleotide represented in SEQ ID NO: 5 tile fragment represented in SEQ ID NO: 11 is produced.

SEQ ID NO: 7 is the sequence of a synthetic oligonucleotide encoding a portion of the Large-T antigen from SV-40. When used in a PCR reaction with the oligonucleotide represented in SEQ ID NO: 8 the fragment represented in SEQ ID NO: 12 is produced.

SEQ ID NO: 8 is the sequence of a synthetic oligonucleotide encoding a portion of Large-T antigen from SV-40. When used in a PCR reaction with the oligonucleotide represented in SEQ ID NO: 7 the fragment represented in SEQ ID NO: 12 is produced.

SEQ ID NO: 9 is the nucleotide sequence portion of the 35-S CaMV promoter region amplified by primers represented in SEQ ID NOs: 1 and 2. This fragment has a melting temperature of 83-87.5° C.

SEQ ID NO: 10 is the nucleotide sequence portion of the soybean lectin gene LE-1 that is amplified by primers represented in SEQ ID NOs: 3 and 4. This fragment has a melting temperature of 81.5-83.5° C.

SEQ ID NO: 11 is the nucleotide sequence portion of a unique genome locus from Escherichia coli 0157:H7 that is amplified by primers represented in SEQ ID NOs: 5 and 6. This fragment has a melting temperature of 82.6-89° C.

SEQ ID NO: 12 is the nucleotide sequence portion of the SV-40 Large-T antigen amplified by primers represented in SEQ ID NOs: 7 and 8. This fragment has a melting temperature of 77-79° C.

### DESCRIPTION OF THE INVENTION

The inventive, PCR-based method detects and quantifies double stranded nucleic acid molecule (“dsDNA” or “target”) by monitoring the fluorescence of the amplified target (“target amplicon”) during each amplification cycle at selected time points.

As is well known to the skilled artisan, the two strands of a dsDNA separate or melt, when the temperature is higher than its melting temperature. Melting of a dsDNA molecule is a process, and under a given solution condition, melting starts at a temperature (designated T_{MS} hereinafter), and completes at another temperature (designated T_{ME} hereinafter). The familiar term, Tm, designates the temperature at which melting is 50% complete. For the inventive methods, the melting curve characteristics of a target amplicon is predetermined.

A typical PCR cycle involves a denaturing phase where the target dsDNA is melted, a primer annealing phase where the temperature optimal for the primers to bind to the now-single-stranded target, and a chain elongation phase (T_{E}) where the temperature is optimal for DNA polymerase to function. According to the present invention, T_{MS} should be higher than T_{E}, and T_{M} should be lower (often substantially lower) than the temperature at which the DNA polymerase is heat-inactivated. Melting curve characteristics are, of course, the intrinsic properties of a given dsDNA molecule. A desirable melting curve is usually achieved by selecting the length and/or GC content of the target amplicon. Melting curve characteristics may also be altered by changing the PCR primers that are used to amply them. For example, adding GC-rich overhangs to the 5′ end of the primers will increase the Tm for the amplified target.

Double stranded nucleic acid molecules exhibits fluorescence under ultraviolet light when they are associated with certain dyes, and the intensity of the fluorescence may be proportionate to concentration of the dsDNA. Methods taking advantage of such relationship to detect and quantify dsDNA are known in the art. Many dyes are known and used in the art for these purposes. The instant methods also takes advantage of such relationship. An example of such dyes includes intercalating dyes. Examples of such dyes include, but are not limited to, SYBR Green-I®, ethidium bromide, propidium iodide, TOTO®-1 {Quinolinium, 1-1′-[1,3-propanediylbis[(dimethyliminio)-3,1-propanediyl]]bis[4-[(3-methyl-2(3H)-benzothiazolylidene)methyl]]-,tetraiodide}, and YoPro® {Quinolinium, 4-[(3-methyl-2(3H)-benzoxazolylidene)methyl]-1-[3-(trimethylammonio)propyl]-,diiodide}. Most preferred dye for the instant invention is a non-asymmetrical cyanide dye such as SYBR Green-I®, manufactured by Molecular Probes, Inc. (Eugene, Oreg.). The SYBR Green®/DNA complex, and SYBR Green® alone, has an inherent temperature dependent fluorescence. As the temperature increases, the fluorescence of the SYBR Green®/DNA complex will naturally decrease, even though the dsDNA strands are not separated. The rate of change increases proportionally to the concentration of DNA. In order to distinguish this temperature-dependent change from the change due to dsDNA strand separation, it is necessary that a threshold to be established before, and during, the measurements surrounding each amplicon in a reaction.

According to the instant invention, for each PCR cycle, the fluorescence (F) of the PCR reaction mixture is measured immediately before the temperature starts to increase from T_{E} (F_{E} at T_{E}), at any temperature point (T_{B}) in between T_{E} and T_{MS} (F_{B} at T_{B}), immediately below the starting temperature of melting (F_{MS} at T_{MS}) and immediately above the completion of melting (F_{ME} at T_{ME}).

Depending on the instruments used in the PCR reactions and in the measurements of the various fluorescence values, the temperature may need to be held steady for a period of time when making the various fluorescence measurements. For example, when a Perkin-Elmer 7700 Sequence Detection System is used, any time periods between 0.5 to 60 seconds are suitable. Preferably, a period of between 1-45 seconds, more preferably between 1-30 seconds, and still more preferably between 1-15 seconds are suitable. The most preferred time period for make a fluorescence measurement is 7 seconds.

From these values, a baseline slope (S_{B}), is calculated. S_{B} is defined by negative (F_{B} minus F_{E}), divided by (T_{B} minus T_{E}), and a melting phase slope (S_{M}), defined by negative (F_{ME} minus F_{MS}) divided by (T_{ME} minus T_{MS}), is also calculated.

As the PCR amplification proceeds, the concentration of the target amplicon increases and so does the value of F_{MS}. The number of amplification cycles (the “threshold cycle number, C_{T}”) it takes for the first appearance of a positive change in the slope, where the quantity (S_{M}−S_{B}) is greater than zero, is correlated with the starting concentration of the target amplicon (C).

A standard curve for the target amplicon is established by starting with a series of dilutions of a solution of the target amplicon whose concentration is known. By repeating the method above for each concentration in the dilution series, under identical PCR conditions, C_{T} is determined for each of the known concentration. The standard curve for the target amplicon under a given PCR condition is thus established by plotting C_{T} against C. Preferably, the standard curve plots a suitable range of concentrations between 1-10⁹ copies of the target amplicon, preferably a range of 10-10⁸, more preferably 10-10⁷, particularly preferably 10-10⁶ copies.

In order to determine the concentration of the target amplicon in a sample suspected of containing the target amplicon, the sample is prepared in a suitable way such that it is suitable for amplification by PCR. The sample is then subject to a PCR amplification under the identical conditions under which its corresponding standard curve is established. The value of C_{T} for the sample is determined as discussed above and is compared with the standard curve to establish the corresponding concentration.

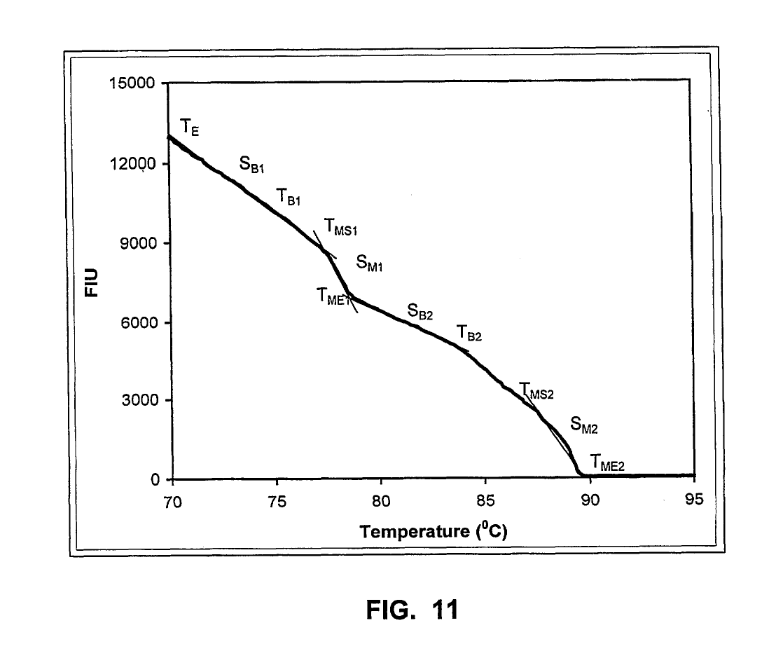

The method according to the instant invention can also be used to detect simultaneously multiple target amplicons (“multiplex detection”). Referring to FIG. 11, it is apparent that, when the sample contains more than one target amplicon, the respective S_{B} and S_{M} for each amplicon, F_{E} at T_{E} and F_{B} at T_{B} must be determined. Accordingly, for multiplex detection, the target amplicons should have distinguishable melting curve characteristics, such that F_{E} at T_{E} and F_{B} at T_{B} for each amplicon is determinable.

If the sample contains two target amplicons (designated a first target nucleic acid molecule and a second target nucleic acid molecule), the melting of the first target nucleic acid molecule starts at a temperature T_{MS1} and completes at a temperature T_{ME1}, the melting of the second target nucleic acid molecule starts at a temperature T_{MS2} and completes at a temperature T_{ME2}. According to the instant invention, T_{MS2} is greater than T_{ME1}. It is recognized that under usual conditions, T_{ME1} is not lower than 55° C., while T_{MS2} is not higher 95° C., and a 3-5° C. difference is usually sufficient for multiplex amplification and quantification. To quantify the starting concentration of a target amplicon in a multiplex PCR reaction, amplicons (including internal standard controls, other target amplicons, and non-specific products, e.g. primer dimers) must be designed or selected to have non-overlapping melting temperatures with other possible products in the reaction. This is to ensure that the products melt at different temperatures, and the various fluorescence values can be differentiated and analyzed independently. The total fluorescence is additive and remain correlated to the concentrations of dsDNA products. This is to say that after a product dissociates or melts, it no longer contributes to the total florescence.

The chain extension temperature (T_{E}), however, is identical for both amplicons. A standard curve for each of the target nucleic acid molecule is established by: simultaneously PCR-amplifying, in the presence of a suitable fluorescent dye, the target nucleic acid molecules with a known starting concentration (C₁ and C₂) as provided above. Specifically, the fluorescence (F) is measured during each amplification cycle at the temperature immediately before the temperature starts to increase from T_{E} (F_{E} at T_{E}), at any temperature point (T_{B1}) in between T_{E} and T_{MS1} (F_{B1} at T_{B1}), at T_{MS1} (F_{MS1} at T_{MS1}), at T_{ME1} (F_{ME1} at T_{ME1}), at any time point (T_{B2}) in between T_{ME1} and T_{MS2} (F_{B2} at T_{B2}), at T_{MS2} (F_{MS2} at T_{MS2}), at T_{ME2} (F_{ME2} at T_{ME2}); a baseline slope is calculated for the first target molecule (S_{B1}), defined by negative (F_{B1} minus F_{E}), divided by (T_{B1} minus T_{E}), and a first amplicon melting phase slope is calculated for the first molecule (S_{M1}), defined by negative (F_{ME1} minus F_{MS1}) divided by (T_{ME1} minus T_{MS1}); and a baseline slope for the second target molecule (S_{B2}=−(F_{B2} minus F_{ME1})/(T_{B2} minus T_{ME1})) and a melting phase slope for the first molecule (S_{M2}=−(F_{ME2} minus F_{MS2}) /(T_{ME2} minus T_{MS2}) are similarly determined. The number of PCR cycles (N₁) required for the quantity (S_{M1} minus S_{B1}) to first become greater than zero; and the number of PCR cycles (N₂) required for the quantity (S_{M2} minus S_{B2}) to first become greater than zero, are recorded. These steps are repeated) for a suitable range of concentrations of interest for each of the two target molecules; and C₁ is plotted against N₁ to obtain a standard curve for the first target molecule; and C₂ is plotted against N₂ to obtain a standard curve for the second target molecule.

The above steps are then repeated for a sample suspected of containing an unknown concentration of the first and second target nucleic acid molecules, to obtain an N₁ value and an N₂ value for the sample, and to determine the target nucleic acid molecule concentrations via the standard curves.

Fluorescence intensity fluctuates inherently from cycle to cycle. This constitutes the background noise. In order to eliminate or at least minimize such a noise, and also to achieve instrument-to-instrument consistency, a threshold value is set arbitrarily. Any fluorescence level below such a threshold level is ignored and a determination is made that no meaningful or specific amplification is considered to have occurred. Only fluorescence level above the threshold level is measured and used for detection and quantification purposes.

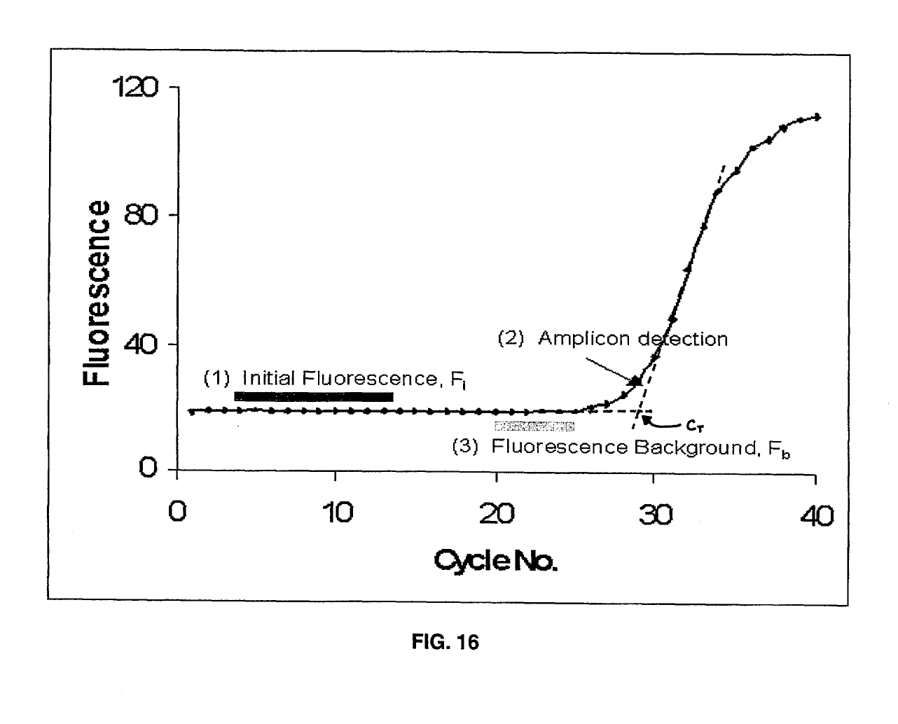

There is typically no detectable product during early cycles of PCR; therefore the first few cycles can be used to determine the threshold value. According to the instant invention, an Initial Fluorescence, F_{I}, is defined as the average fluorescence for the first few PCR cycles (FIG. 16). According to the most preferred embodiment, initial cycles #4 to 12 are used. Threshold Fluorescence, F_{B}, is the average fluorescence signal from cycle N-10 to cycle N-5 prior to exponential phase of PCR as threshold background signal. The detectable level of amplicon is pre-determined to be at cycle N when the delta slope (S_{M}−S_{B}) fluorescence was 67% above baseline. In the exponential growth phase of product, the value of Ln[(Fₙ−F_{B})/F_{B}] is a linear function of the cycle number. The slope and R² for a linear regression is determined for F values in the range of 1.06 F_{B} to 1.67 F_{B}. The intersection of the regression line and the threshold fluorescence of 1.67 F_{B} (Ln=−0.4) is the cycle threshold, C_{T}, for the amplification event. Plotting the log of DNA concentration against C_{T} then created a standard curve Examples 1, 2, and 6 demonstrate this embodiment.

According to another embodiment of the present invention, a threshold may also be the average change in fluorescence (delta slope of S_{M}−S_{B}) for the initial 10 cycles (#4 to 13) plus ten times the standard deviation of these values. Initial Fluorescence, F_{I}, was defined as the average fluorescence for initial cycles #4 to 12. The number of cycles (Ct) it takes for the first appearance above the threshold that is the sample's threshold cycle (Ct). Examples 3, 4, and 5 demonstrates this method.

In another preferred embodiment, the multiplex detection method of the instant invention is used to determine the copy number per genome of a target nucleic acid (Target A), using another target nucleic acid (Target B) as a reference point, wherein the copy number/genome for Target B is known. Because Target A and Target B reside in the same genome, and the copy number/genome for Target B is known, when Target A and Target B are co-amplified and quantified using the multiplex detection method of the instant invention, the Target A copy number/genome can be readily calculated from the ratio between the amount of Target A and the amount of Target B, without the need of knowing or determining the genome size or the need to quantify the amount of genomic DNA used in the starting sample. An example of this embodiment is provided in Example 2.

The instant detection method can be used to detect and quantify any target dsDNAs, from which the presence and level of target organisms can be determined. Examples of target organisms include pathogenic organisms including fungi, bacteria, infectious animals, viruses etc. Particularly, the instant methods have been applied in the detection of Salmonella typhimurium, Salmonella enteriditis, Escherichia coli O157:H7, Listeria spp., Listeria monocytogenes, Cryptosporidium parvum, Campylobacter jejuni, Campylobacter coli, Staphylococcus aureas, Pseudomonas aeruginosa, and SV-40 viral DNA. The instant methods can also be used for other clinical or non-clinical uses. For example, the methods can be used to determine the presence of genetically modified organisms in foods or feeds. Sequences such as 35S CaMV promoter (a sequence found in Roundup Ready® soybeans), and the Cry9C gene (found in the StarLink Corn, Hua et al. (2001) Appl Environ Microbiol 67:872-879) have been detected and quantified using the instant methods. Other common transgenes include, but are not limited to, EPSPS (5-enolpyruvylshildmate-3-phosphate synthase) as a glyphosphate tolerant gene (Ye et al. (2001) Plant J 25:261-270), the phosphoenolpyruvate carboxylase (PEPC) promoter (in BT176 Corn); the hsp70 promoter of Cry1A(b) gene (found in Mon 80100 corn) Cry1A(b) gene (Mon 809 corn); NOS gene (Mon810 Corn), and the actin promoter gene (GA21 Corn). As internal positive and/or copy number control, the lectin gene, the invertase gene, and the aldolase gene may all be used, where appropriate.

The instant method is very specific and sensitive. As few as 10 copies of the target dsDNA are detected.

In a preferred embodiment the PCR tablet for pathogenic organisms contains an internal positive control. The advantages of an internal positive control contained within the PCR reaction have been previously described (PCT Application No. WO 97/11197 published on Mar. 27, 1997, the contents of which are hereby incorporated by reference) and include (i) the control may be amplified using a single primer; (ii) the amount of the control amplification product is independent of any target DNA contained in the sample; (iii) the control DNA can be tabletted with other amplification reagents for ease of use and high degree of reproducibility in both manual and automated test procedures; (iv) the control can be used with homogeneous detection, i.e., without separation of product DNA from reactants and (v) the internal control has a melting profile that is distinct from other potentially produced amplicons in the reaction. Control DNA will be of appropriate size and base composition to permit amplification in a primer directed amplification reaction. The control DNA sequence may be obtained from the target bacteria, or from another source, but must be reproducibly amplified under the same conditions that permit the amplification of the target amplicon DNA. In a preferred embodiment, the control DNA is similar in size and base composition to the target DNA to be detected. For example, a control nucleic acid fragment was isolated from the genus Salmonella and was identical to the target to be detected, except that it was engineered to allow for amplification with a single primer (WO 97/11197). The control DNA is useful to validate the amplification reaction. Amplification of the control DNA is accomplished concurrently with the test sample containing the target DNA. Within the context of the present invention a sample is subjected to the test PCR procedure in parallel with a control containing the control DNA as well as the sample. If the control shows amplification, there is positive indication that the procedure has been effective regardless of the positive or negative results attained in the parallel test. In order to achieve significant validation of the amplification reaction a suitable number of copies of the control DNA must be included in each amplification reaction. It is well known that sample matrix components, including food, can cause inhibition of PCR and therefore a resulting decrease in product formation and signal. Alternatively, the presence of certain food components in the PCR reaction has also been found to result in the opposite result, i.e. enhancement of the signal when fluorescent dye detection is employed. Use of the control as described herein eliminates such false positive results. Moreover, by calibrating the level of response in the control, it is possible to evaluate and compensate for any suppression or enhancement of the reaction in the test caused by extraneous material such as is found in many food-derived matrices.

In addition to being used in connection with PCR, the instant method may also be used with other nucleic acid amplification methods such as strand displacement, ligase chain reaction(LCR) and nucleic acid sequence based amplification (NASBA) (See e.g. Food Microbiology Fundamentals and Frontiers, 1997, M. E. Doyle, L. R. Beucha, and T. J. Mondville, ASM Publication, pp. 723-724).

According to a preferred embodiment, an automated thermal cycler with fluorescence detection capabilities such as the Perkin-Elmer 7700 Sequence Detection System available from the Perkin-Elmer Corporation is used. Fluorescence data are exported and processed with the help of a data processing device such as a personal computer, with various transformations when necessary. Methods and instruments for such automated operation are apparent to a skilled person and are exemplified in the examples that follow.

### EXAMPLE 1

Single Target Q-PCR Assay—Detection of Genetically Modified DNA by Targeting the 35S—CaMV Promoter

The detection of genetically modified (GM) crops is becoming more important because of food production and consumer concerns, and the concomitant legal issues. We developed a rapid DNA extraction method and combined it with a homogeneous PCR-based assay that uses fluorescence detection for identifying and/or quantifying genetically modified material in soybeans, maize kernels, and a variety of processed samples. The presence of GM DNA was determined using a pair of primers directed towards the 35S viral promoter. This pair of primers was designed Franck et al.

These primers amplify a 206 bp fragment of the CaMV 35S promoter sequence, which is present in nearly all genetically modified organisms and thus used to screen samples for the GM product. Soybeans that have been genetically modified to be resistant to Roundup, a widely used herbicide, are referred to as “Roundup Ready”, meaning that by definition they contain GM material.

The closed-tube homogeneous PCR process described below uses a commercial detection system and DNA intercalating dye, SYBR Green-I. During each thermal cycle, fluorescence data is collected at an intermediate temperature between the extension and denaturation steps. As the specific PCR product is generated, the dye intercalates into the product and the total fluorescence signal increases. The fluorescence value of the intercalating dye is inversely proportional to the temperature. We compare the change in slope of the fluorescence value of a specific amplicon with the baseline slope of intercalating dye. Then we record the thermal cycle at which the first appearance of a positive change in slope occurs, where the amplicon slope is greater than the baseline slope.

Standards with known levels of genetically modified 35S promoter DNA (ranging from 35 to 4375 copy of genome per PCR) are amplified and the fluorescence signal recorded after each cycle. A curve is generated based on the linear regression fit of the threshold curve (C_{T}) versus the log of percent genetically modified material. Unknown sample C_{T} values are plotted against the standard curve and a genetically modified percentage is determined. Using a similar technique, the amount of total soy DNA is quantified by targeting an amplification reaction to the lectin-coding region of soybean DNA. The ratio of the amount of the modified insert to the total amount of soy DNA allows an accurate percentage of genetically modified material to be calculated.

Material and Methods

Extraction of Standard GMO Calibrator DNA

Materials (DNA Extraction)

Qiagen™ Plant DNeasy mini column kit, Qiagen Inc. (Valencia, Calif.)

100% ethanol

DNA elution buffer: 30 mM Tris/0.1 mM VDTA, pH 8.35

GM-Roundup Ready™ Certified Reference Material IRMM410 (dried soy bean powder) (GM-RR™) (Fluka, Retieseweg, Belgium)

Unknown samples from Protein Technologies International (St. Louis, Mo.)

C9K-BQAP 91 (wheat flour)

C9K-BPW 145 (isolated soy protein, lecithin)

E9J-BE 0122 (isolated soy protein)

Method

Extract one large pool of 2% RRTM soy GM-standard DNA (undiluted, as level 1 DNA calibrator), using the manufacturer's recommendations:

Weight out 30 (±3) mg of 2% GM-RR™ soy protein powder per sample (or 30 mg unknown % GMO sample) and transfer to 10 microcentrifige tubes. For each tube, add 400 μl of Buffer AP1 (supplied with kit) and 4 μl of RNase stock solution to above sample and vortex vigorously. Incubate the mixture for 10 min at 65° C. in a water bath. Mix 2-3 times during incubation by inverting the tube.

Add 130 μl of Buffer AP2 (supplied) to each tube of the mixture, mix, and incubate for 5 min on ice. Centrifuge the mixture for 5 min in a tabletop centrifuge at 6000×g to pellet the aggregated protein debris.

Transfer the top layer of clear solution to a new set of tubes without disturbing the protein debris pellet (˜400 μl supernatant per sample). The object is to get most supernatant volume possible; some sample may not pellet well. In that case, centrifuge one more time.

Add 200 μl of Buffer AP3 (supplied) to each tube of supernatant. Add 400 μl of 100% ethanol and mix by repeatedly by pipetting up and down or by vortexing the vial.

Place a DNeasy mini-column (supplied) in a 2-ml collection tube. Apply each 650 μl tube of the mixture. Centrifuge for 1 min at >6000×g and discard the flow-though. Repeat for the remaining samples. Discard the flow-though and collection tube.

Place the DNeasy column in a new 2-ml collection tube, add 500 μl Buffer Aw (supplied) into each of the columns and centrifuge for 1 min at >6000×g. Discard the flow-though and reuse the collection tube in the next step.

Add 500 μl Buffer AW to each column and centrifuge for 2 min at maximum speed to dry the DNeasy membrane.

Place the DNeasy column into a clean 1.5 ml microcentrifuge tube and pipette 200 μl of pre-heated (65° C.) 30 mM Tris/0.1 mM EDTA, pH 8.35 elution buffer directly into each of the columns. Incubate for 5 min at room temperature and then centrifuge for 2 min at >6000×g to elute the DNA.

Pool all 10 eluted reference standards DNA samples together. Using gel filtration HPLC to quantify the purified soy DNA concentration.

HPLC Process to Quantify DNA Concentration

Gel filtration HPLC was performed with an aqueous buffer (0.1 M phosphate/0.3 M NaCl, Ph 7.0) as mobile phase at flow rate of 1 ml per minute. Quantifying DNA fragment concentration, by injecting known amount of 1000 base pair pure DNA (from 5 to 500 ng) per assay, then, plot the amount (ng) of DNA Vs HPLC peak area (mAU) to create a calibration curve. Use this calibration curve to determine the unknown sample DNA concentration.

Adjust the total DNA concentration to be 6 ng/ul as level 1 calibrator. Then, make a serial dilution of the 2% RR™ DNA (L1) into 0.4% (⅕ dilution, L2), 0.08% (1/25 dilution, L3), and 0.016% GMO (1/125 dilution, L4), with DNA elution buffer.

Use the same set of GMO DNA standards for both CaMV (2, 0.4, 0.08 and 0.016%) and lectin (100, 20, 4, and 0.8%) respectively.

Based on the reference described by Arumuganathan et al that soybean DNA average size is around 1.15E+9 base pair. The molecular mass of genomic soy DNA is about 7.475E11 Dalton. We calculate the DNA copy number for CaMV and Lectin Level 1 to Level 4 per PCR are 4375, 875, 175, and 35 for CaMV and 212, 500, 42, 500, 8500, and 1700 for Lectin soy DNA respectively.

PCR Reagent and Process

Materials (PCR Reagent)

RiboPrinter® System deionized water (Qualicon, Inc., Wilmington, Del.)

25 mM mgCl₂ (Perkin-Elmer, Branchburg, N.J.)

10× PCR Buffer=100 mM Tris/500 mM KCl/0.01% Gelatin, pH 8.3 (Perkin-Elmer)

Primers (Trilink Biotechnologies Inc., San Diego, Calif.)

CaMV Primer P-93: 25-mer 5′(CGA AGG ATA GTG GGA TTG TGC GTC A) 3′.CAMV1-25-93

CaMV Primer rc-290 :25-mer 5′(AAG GTG GCT CCT ACA AAT GCC ATC A) 3′.CaMV 1-25-rc290

SYBR Green I Intercalating dye (Molecular Probes, Eugene, Oreg.)

Bovine Serum Albumin (Roche Molecular Biochemicals, Indianapolis, Ind.)

Reagent tablet Qualicon, Inc., Wilmington, Del.):

1.2 μM SYBR green I, 4 mg/tablet BSA, all four d-NTP, 1.5 units Taq™ polymerase

Hardware:

PE/ABI PRISM 7700 Sequence Detection System (Perkin-Elmer, Foster City, Calif.)

Method

Pre-mix 2× of PCR buffer with 1 mM Mg⁺² and 2× of CaMV Primers together to obtain a 2× working concentration (20 mM Tris/100 mM KCl/4 mM MgCl₂/300 nM of CaMV primers). To 25 μl of this mixture, add 25 μl of each sample of extracted DNA.

Final PCR Buffer Concentrations:

25 mM Tris/50 mM KCl/0.001% gelatin/0.05 mM EDTA, pH 8.3

2 mM Mg⁺² (total including contribution from PE Buffer)

150 nM of each CaMV primer

Quantitative PCR Assay

Pipette 25 μl of each the five levels of DNA standards (triplicate) and the unknown sample extract into a PCR well (triplicate) which contains one reagent tablet. To 25 μl of each sample of extracted DNA, add 25 μl of the CaMV /buffer mixture.

Place the sample tubes into a cooling block (Qualicon, Inc.) and vortex the PCR tubes to mix the sample, reagent, and tablet. Place the rack of PCR tubes into PE/ABI PRISM 7700 Sequence Detection System (Perkin-Elmer).

PCR Parameter Set up

| | |
|---|---|
| Stage I: | 94° C. for 3 minutes |
| Stage II: | Run 40 cycles with: |
| | 94° C. for 20 seconds |
| | 70° C. for 40 seconds |
| | 72° C. for 1 minute |
| | 72° C. for 7 seconds |
| | 82.5° C. for 7 seconds |
| | 83.5° C. for 7 seconds |
| | 87.6° C. for 7 seconds |
| Stage III: | 72° C. for 3 minutes |

Collect the fluorescence signal from stage II at (T_{E}) 72° C., (T_{B}) 82.5° C., (T_{MS1}) 83.5° C., (T_{ME1}) 87.6° C. in order to quantify the copy number of CaMV DNA sequences.

Data Process and Analysis

Measure the fluorescence excited by the beam during each amplification cycle: At the temperature of the end of extension phase (F_{E} at T_{E}: 72° C.), before amplicon start melting (F_{B1} at T_{B1} 82.5° C.), beginning the melting temperature of the amplified 35S CaMV PCR product (F_{MS1} at T_{MS1} 83.5° C.) and at the end of the melting temperature of the amplified CaMV amplicon (F_{ME1} at T_{ME1}: 87.6° C.).

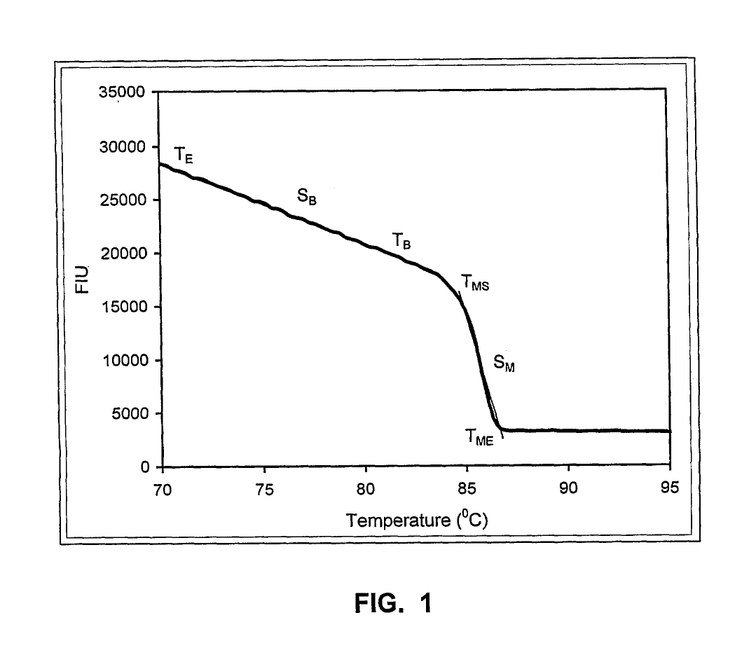

Determine the change in slope from a baseline slope (S_{B1}), defined by the negative value of (F_{B1} minus F_{E}) divided by (T_{B1} minus T_{E}), to amplicon 35S CaMV melting phase slope (S_{M1}), defined by the negative value of (F_{ME1} minus F_{MS1}) divided by (T_{ME1} minus T_{MS1}). See FIG. 1. Record the thermal cycle at which the first appearance of a positive change in slope occurs, where (S_{M1} minus S_{B1}) is greater than zero. Repeat the steps above 40 times to determine a range of concentrations from 35 to 4375 genomic copies of CaMV as the target start concentration, to provide the standard curve. Quantify the starting concentration of GMO in an unknown sample by running the same DNA extraction and PCR process described above. Then, compare the resultant thermal cycle number with the standard curve to determine the starting GMO concentration in unknown sample.

Results

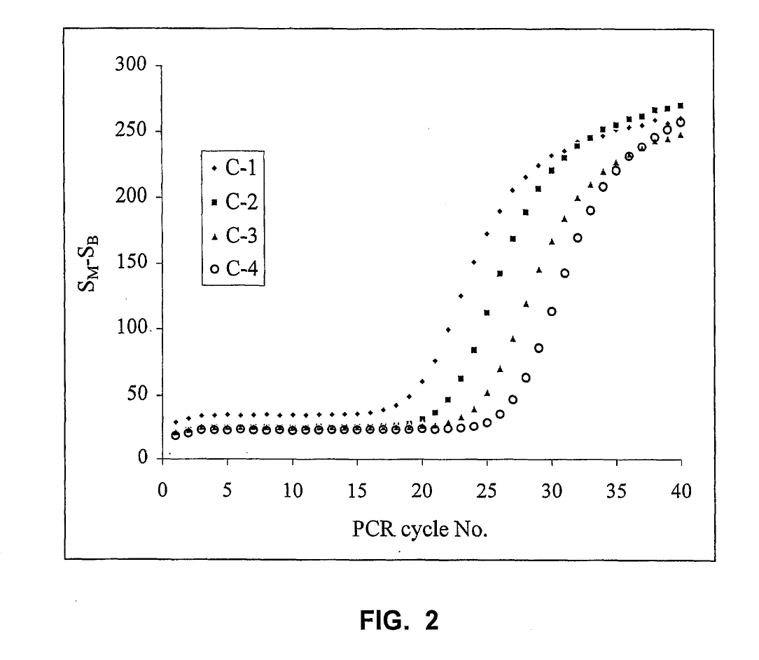

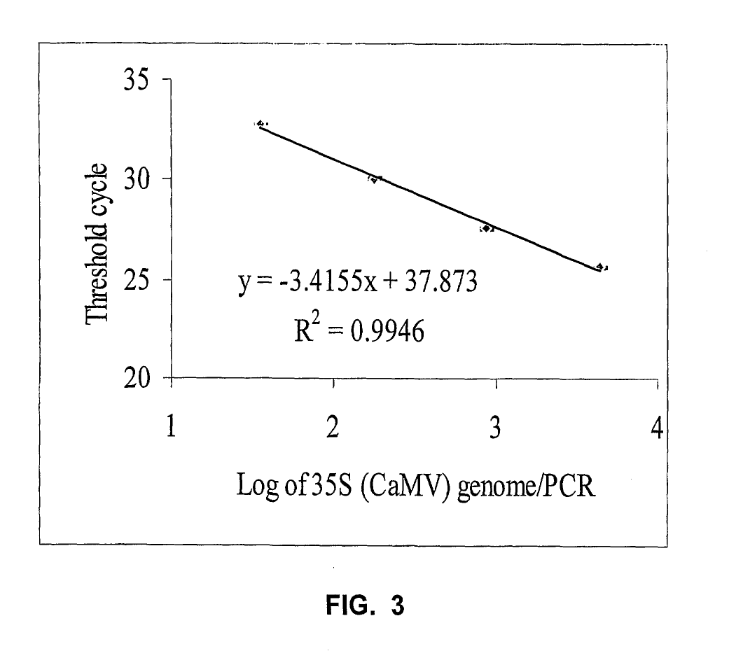

See FIG. 1 Melting Profile of CaMV Amplicon, FIG. 2 Real-time Quantitative PCR for CaMV, and FIG. 3 CaMV Standard Calibration Curve.

Sample #COC-BXJ539 (FUJI protein 545) first time appear at cycle 33.63, its CaMV amplicon melting slope is greater than baseline slope. Based on the linear regression from calibration curve of CaMV, the sample contents 18 coy of 35S CaMV promoter DNA in it.

#M35-490 (isolated soy protein,) first time appear at cycle 30.72, its CaMV amplicon melting slope is greater than baseline slope. Based on the linear regression from calibration curve of CaMV, the sample contents 125 copy of 35S CaMV promoter DNA in it.

#NAHX-61509 (soy flake) first time appear at cycle 33.35, its CaMV amplicon melting slope is greater than baseline slope. Based on the linear regression from calibration curve of CaMV, the sample contents 21 copy of 35S CaMV promoter DNA in it.

Reference

Franck A, Guilley, H., Jonard, G., Richards, K., and Hirth, L. Nucleotide Sequence of Cauliflower mosaic virus DNA. Cell 21(1): 285-294 (1980).

Arumuganathan, K and Earle, e. D. (1991) Nuclear DNA content of some important plant species, Plant Molecular Biology Reporter 9(3): 211-215. Tablet I.

### EXAMPLE 2

Single Target W-PCR Assay—Detection of endogenous Plant Genes and Their use as Genome Copy Number References

We developed a rapid DNA extraction method and combined it with a homogeneous PCR-based assay that uses fluorescence detection for identifying and/or quantifying genetically modified material in soybeans, maize kernels, and a variety of processed samples. The percentage of genetically modified material not only depends on the presence of 35S CaMV DNA, but requires determination of how many copies of total soy DNA had been extracted in each sample. A natural marker, lectin, in almost every green plant was selected as a control factor. A pair of primers was designed by Vodkin et al. to amplify a 186 bp fragment of the lectin gene.

Material and Methods

Extraction of GMO Standard Calibrator DNA

Same process as described in Example I.

PCR Reagent and Process

Materials (PCR Reagent)

RiboPrinter® System deionized water (Qualicon, Inc., Wilmington, Del.)

25 mM MgCl₂ (Perkin-Elmer, Branchburg, N.J.)

10× PCR Buffer=100 mM Tris/500 mM KCl/0.01% Gelatin, pH 8.3 (Perkin-Elmer)

Primers (Trilink Biotechnologies Inc., San Diego, Calif.)

Lectin Primer P-1423: 5′(CAA CGA AAA CGA GTC TGG TGA TCA AGT) 3′.

Lectin -27-1423

Lectin Primer rc1555: 5′(TGG TGG AGG CAT CAT AGG TAA TGA GAA) 3′.

Lectin -27-rc1555

SYBR Green I Intercalating dye (Molecular Probes, Eugene, Oreg.)

Bovine Serum Albumin (Roche Molecular Biochemicals, Indianapolis, Ind.)

Reagent tablet (Qualicon, Inc., Wilmington, Del.):

1.2 μM SYBR green I, 4 mg/tablet BSA, all four d-NTP, 1.5 units Taq™ polymerase.

Hardware:

PE/ABI PRISM 7700 Sequence Detection System (Perkin-Elmer, Foster City, Calif.)

Method

Pre-mix 2× of PCR buffer with 1 mM Mg⁺² and 2× of Lectin Primers together to obtain a 2× working concentration (20 mM Tris/100 mM KCl/4 mM MgCl₂/400 nM of lectin primers). To 25 μl of this mixture, add 25 μl of each sample of extracted DNA.

Final PCR buffer Concentrations:

25 mM Tris/50 mM KCl/0.001% geltin/0.05 mM EDTA, pH 8.3

2 mM Mg⁺² (total including contribution from PE Buffer)

200 nM of each Lectin primer

Quantitative PCR Assay

Pipette 25 μl of each the four levels (L1, L2, L3, and L4) of DNA standards (triplicate) and the unknown sample extract into a PCR well (triplicate) which content one reagent tablet. To 25 μl of each sample of extracted DNA, add 25 μl of the 2× lectin/buffer mixture.

Place the sample tubes into a cooling block (Qualicon, Inc.) and vortex the PCR tubes to mix the sample, reagent, and tablet. Place the rack of PCR tubes into PE/ABI PRISM 7700 Sequence Detection System (Perkin-Elmer)

PCR Parameter Set up

| | |
|---|---|
| Stage I: | 94° C. for 3 minutes |
| Stage II: | run 40 cycles with: |
| | 94° C. for 20 seconds |
| | 70° C. for 40 seconds |
| | 72° C. for 1 minute |
| | 72° C. for 7 seconds |
| | 81° C. for 7 seconds |
| | 83.5° C. for 7 seconds |
| Stage III: | 72° C. for 3 minutes |

Collect the fluorescence signal from stage II at (T_{E}) 72° C., (T_{B}) 81° C., (T_{MS1}) 81° C. (T_{ME1}) 83.5° C. for quantify the copy number of lectin DNA.

Data Process and Analysis

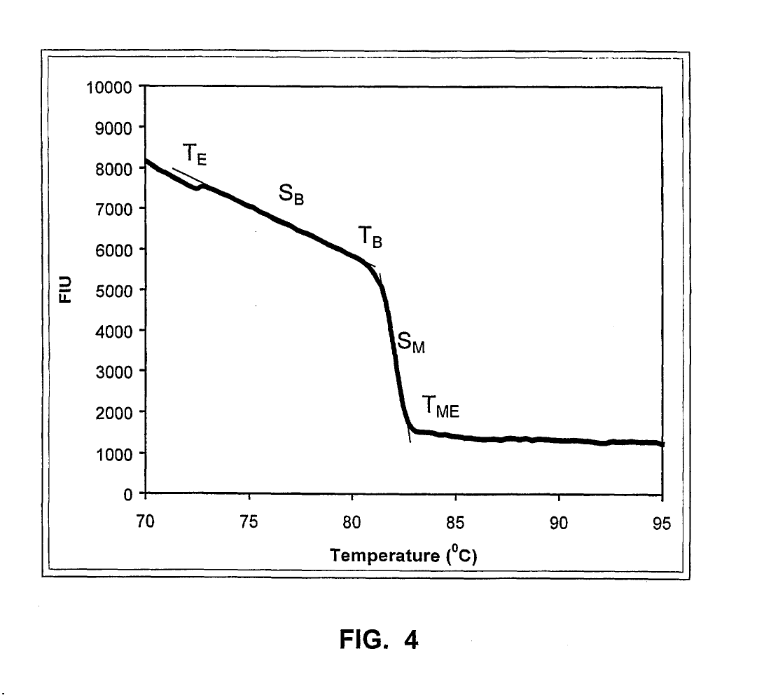

Measure the fluorescence excited by the beam during each amplification cycle: At the temperature of the end of ex-tension phase (F_{E} at T_{E}: 72° C.), before amplicon start melting (F_{B1} at T_{B1}: 81° C.), beginning the melting temperature of the amplified lectin PCR product (F_{MS1} at T_{MS1}: 81° C.) and at the end of the melting temperature of the amplified lectin PCR product (F_{ME1} at T_{ME1}: 83.5° C.). See FIG. 4.

Record the thermal cycle at which the first appearance of a positive change in slope occurs, where (S_{M1} minus S_{B1}) is greater than zero. Repeat the above steps 40 times to determine a range of concentrations from 1700 to 212500 copy of lectin genome per PCR to provide the standard curve. Quantify the starting concentration of lectin in an unknown sample by running the same DNA extraction and PCR process described above. Then, compare the resultant thermal cycle number with the standard curve to determine the starting lectin DNA concentration in unknown sample.

Results

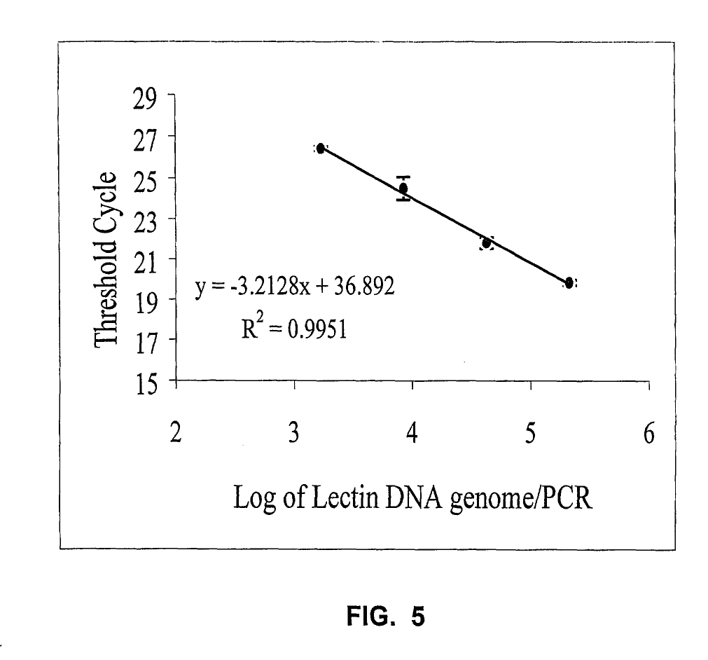

See FIG. 4. Melting Profile of Lectin Amplicon, and FIG. 5. Lectin Standard Calibration Curve.

Sample #COC-BXJ539 (FUJI protein 545) first time appear at cycle 22.55, its Lectin amplicon melting slope is greater than baseline slope. Based on the linear regression from calibration curve of Lectin, the sample contents 29680 copy of Lectin DNA in it.

Based on Example I, the 35S CaMV content in the same sample was 21 copy. The GMO % Content is the ratio of CaMV level to Lectin level and it equals to 0.072%.

#M35-490 (isolated soy protein, lecithin) first time appear at cycle 22.09, its Lectin amplicon melting slope is greater than baseline slope. Based on the linear regression from calibration curve of Lectin, the sample contents 40519 of Lectin DNA in it. Based on Example I, the 35S CaMV content in the same sample was 125 copy. The GMO % Content is the ratio of CaMV level to Lectin level and it equals to 0.31%

#NAHX-61509 (soy flake) first time appear at cycle 23.57, its Lectin amplicon melting slope is greater than baseline slope. Based on the linear regression from calibration curve of Lectin, the sample contents 14880 copy of Lectin DNA in it.

Based on Example I, the 35S CaMV content in the same sample was 18 copy. The GMO % Content is the ratio of CaMV level to Lectin level and it equals to 0.117%.

Reference

Vodkin, L. O., Rhodes, P. R., and Goldberg, R. B. Ca lectin gene insertion has the structural features of a transposable element. Cell 34: 1023-1031 (1983).

### EXAMPLE 3

Single Target Q-PCR Assay—Detection of Bacterial DNA

A homogeneous quantitative assay was developed for bacterial DNA. The assay uses PCR in the presence of SYBR Green I, a DNA intercalating dye. This method can quantify the initial copy number of pathogenic bacteria, e.g., E. coli O157: H7, in the reaction. The method involves collecting data during each thermal cycle of PCR. Fluorescence data is collected at intermediate temperatures between the extension and denaturation steps. As the specific PCR product is generated, the dye intercalates into the product and the total fluorescence signal increases. The fluorescence value of the intercalating dye is inversely proportional to the temperature. We compare the change in slope of the fluorescence value of a specific amplicon with the baseline slope of intercalating dye. Then we record the thermal cycle at which the first appearance of a positive change in slope occurs, where the amplicon slope is greater than the baseline slope.

The cycle at which the fluorescence rises above this value is the threshold cycle (C_{T}). This value is inversely related to the starting target copy number. Standards of known concentration (1.25E+5 to 1.25E+1 E. coli genome/PCR) are run and a standard curve created by plotting the log concentration against C_{T} for the standard samples. The starting copy number of unknown samples is then determined from this standard curve. The method provides a specific and sensitive assay for quantifying starting copy number of a test sample.

Material and Methods

Cell Culture

Strains of Escherichia coli O157:H7 [DD 1977] were inoculated into 10 ml of BHI broth (brain heart infusion; Difco, Detroit, Mich.) and incubated at 37° C. for 24 hours. The cell count of overnight culture was estimated by spread plate enumeration. These cultures typically generated cell densities of approximately ˜1×10⁹ colony-forming units/ml (CFU/ml).

Sample Dilution

Fresh culture was immediately diluted 10-fold with BHI broth. The cell counts of target E. coli O157:H7 in the final were approximately 10⁸, 10⁷, 10⁶, 10⁵, 10⁴, and 0 CFU/ml respectively. One 5 μl sample of each diluted culture was removed and transferred to a lysis tube containing 195 μl of PCR buffer [3 μM SYBR Green I® (Molecular Probes, Inc.)., 200 ng/μl Pronase-E (Sigma Chemical Co., St. Louis, Mo.), 50 mM Tris HCL, 3 mM MgCl₂, 28 mM KCL, 0.1% Triton X 100, pH 8.3]. All lysis tubes were placed in a heating rack set at 37° C. for 20 minutes. The lysed sample tubes were then placed in a 95° C. heating block for 10 minutes to inactivate the Pronase-E. Finally, all samples were subject to PCR amplification followed by fluorescence detection in PE/ABI PRISM 7700 Sequence Detection System to determine the quantity of PCR product from different levels of E. coli O157:H7 for each PCR cycle. The PCR reagents consisted of BAX® for Screening/E. coli O157:H7 tablets(Qualicon, Inc.) that contained proven effective concentrations of 160 mM of DATP, dCTP, dGTP, dTTP, 72 nM of primer 5′(TAC CTG AGG CAG TAG CGA TAA TOA GC)3′.33-26-rc1012; 72 nM of primer 5′(ATG CAG ACC CGC TGG AGT TTG AGA AA)3′.33-26-538 and 1.5 units of Taq™ polymerase. Tablet Lot 9029 E. coli O157:H7 was used for the study.

Quantitative PCR Process

Duplicate aliquots of 50 μL of each lyzed sample were removed and transferred into a PCR tube containing one BAX/E. coli O157:H7 tablet, then amplified in a PE/ABI PRISM 7700 Sequence Detection System.

The reaction proceeded via an initial holding period of 2 min at 94° C., followed by 38 cycles of 94° C./15 seconds and 70° C./2.53 minutes, then, (T_{E}) 70° C., (T_{B1}) 76.5° C./seconds, (T_{MS1}) 82.6° C., (T_{ME1}) and 89.5° C./7 seconds. We collect fluorescence signal from the last 4 events (F_{E} at 70° C./7 sec, F_{B1} at 76.5° C./7 sec, F_{MS1} at 82.6° C./7 sec, F_{ME1} at 89.4° C./7 sec) from every cycle. After the 38 cycles are complete, the PCR tubes are held at 25° C. until analysis.

PCR Product Analysis

The selected primer pair P-538 and rc-1012 amplified a 475-base pair DNA fragment used to identify E. coli O157:H7. Establishment of the threshold cycle value (C_{T}) for distinguishing samples containing various levels of E. coli O157:H7 from those not containing target cells was based on the first appearance of a positive change of the target PCR product melting phase slope (S_{M1}) from the baseline slope (S_{B1}).

Measure the fluorescence excited by the beam during each amplification cycle:

At the temperature of the end of extension phase (F_{E} at T_{E}: 70° C.), before amplicon start melting (F_{B1} at T_{B1}:76.5° C.), beginning the melting temperature of the amplified E. coli O157:H7 PCR product (F_{MS1} at T_{MS1}: 82.6° C.) and at the end of the melting temperature of the amplified E. coli O157:H7 PCR product (F_{ME1} at T_{ME1}:89.4° C.).

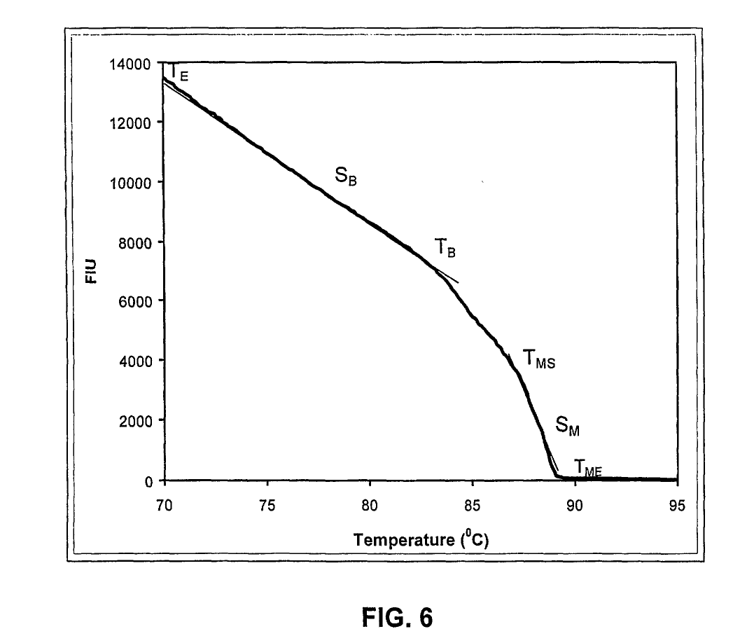

Determine the change in slope from a baseline slope (S_{B1}), defined by the negative value of (F_{B1} minus F_{E}) divided by (T_{B1} minus T_{E}), to amplicon E. coli O157:H7 melting phase slope (S_{M1}), defined by the negative value of (F_{ME1} minus F_{MS1}) divided by (T_{ME1} minus T_{MS1}). See FIG. 6. Record the thermal cycle at which the first appearance of a positive change in slope occurs, where (S_{M1} minus S_{B1}) is greater than zero. Repeat steps 1) through 3) 38 times to determine a range of concentrations from 1.25E+1 to 1.25E+5 of E. coli O157:H7 genome per PCR to provide the standard curve. Quantify the starting concentration of E. coli O157:H7 in an unknown sample by running lysate in the PCR process described above. Then, compare the resultant thermal cycle number with the standard curve to determine the starting E. coli O157:H7 DNA concentration in unknown sample.

Results

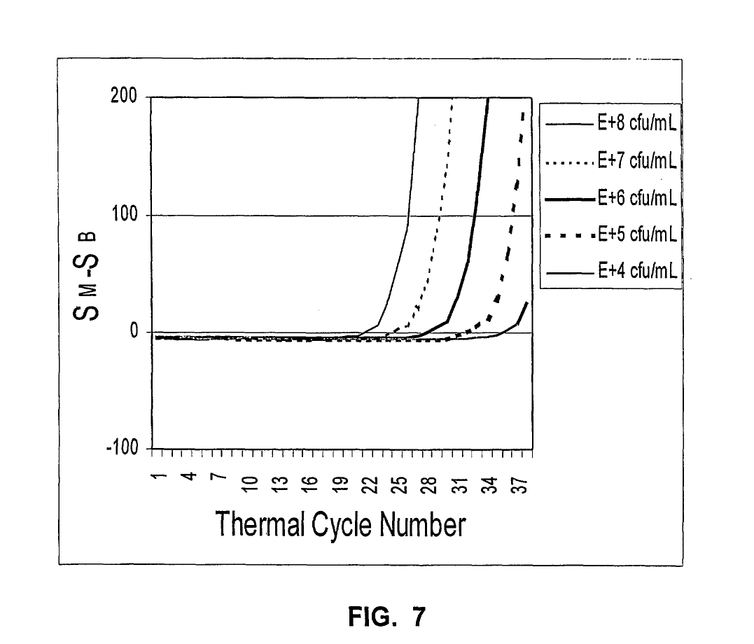

See FIG. 6, Melting Profile of E. coli O157:H7 amplicon, and FIG. 7, E. coli O157:H7 Standard Calibration Curve.

### EXAMPLE 4

Single Target Q-PCR Assay—Detection of Viral DNA

The same basic methodology used for quantifying bacterial DNA was applied to viral DNA in this example.

Material And Methods

SV40 viral DNA was purchased from CIBCO BRL® Life Technology (Rockville, Md.). It is purified from CsCl-banded SV40 virus (Strain 776) Propagated in BSC-1 cells. The molecular weight is about 3.5E6 daltons (5243 base pair dsDNA) supercoiled circular DNA. The SV40 viral DNA was used as a template for PCR amplification in the study.

The stock SV40 DNA concentration was 500 ng/ul, then diluted one to 10,000-fold with distilled, de-ionized water to be 50 pg/ul working stock solution. A serial dilution of SV40 from 50 pg/ul into 20, 10, 4, 0.8, and 0.16 pg/50 ul (correspond to 3.44 E+6, 1.72E+6, 6.88E+5, 1.38E+5 and 2.75E+4 copy DNA) final concentration for PCR reaction.

Finally, all samples were subject to PCR amplification followed by fluorescence detection in PE/ABI PRISM 7700 Sequence Detection System to determine the quantity of PCR product from different levels of SV40 for each PCR cycle. The PCR reagents consisted of BAX® for Screening/E. coli O157:H7 tablets (Qualicon, Inc.) that contained proven effective concentrations of 160 mM of DATP, dCTP, dGTP, dTTP, 72 nM of primer 5′ (TAC CTG AGG CAG TAG CGA TAA TGA GC)3′.33-26-rc1012; 72 nM of primer 5′ (ATG CAG ACC CGC TGG AGT TTG AGA AA)3′.33-26-538 and 1.5 units of Taq™ polymerase. An additional 200 nM of SV40 primer P-4158 5′(TTA AAA AGC TAA AGG TAC ACA ATT TTT GAG CA)-3′ and 200 nM of primer rc-4289 5′(AAA AGC TGC ACT GCT ATA CAA GAA AAT TAT GG)-3′ was added to each PCR reaction. Lot 9020 is tablets were used for the study.

Quantitative PCR Process

Duplicate aliquots of 50 μL of each SV40 DNA sample were removed and transferred into a PCR tube containing one BAX(D for Screening/E. coli O157:H7 tablet, then amplified in a PE/ABI PRISM 7700 Sequence Detection System. The reaction proceeded via an initial holding period of 2 min at 94° C., followed by 38 cycles of 94° C./15 seconds and 70° C./2.53 minutes, then, (T_{E}) 70° C./7 seconds, (T_{B1}) 76.5° C./7 seconds, (T_{MS1}) 77.5° C./7 see, and (T_{ME1}) 79.5° C./7 seconds. Collect fluorescence signal from the last 4 events (F_{E} at 70° C./7 sec, F_{B1} at 76.5° C./7 sec, F_{MS1} at 77.5° C./7 sec, and F_{ME1} at 79.5° C./7 seconds) from each and every cycle. After the 38 cycles were complete, the PCR tubes were held at 25° C. until analysis.

PCR Product Analysis

The selected primer pair P-4158 and rc-4289 was used to amplify a 132-bp of DNA fragment, which can identify the SV40 target DNA. Establishment of the threshold cycle value (CT) for distinguishing samples containing various levels of SV40 from those not containing target cells was based on the first appearance of a positive change of the melting phase slope (S_{M1}) from the baseline slope (S_{B1}).

Measure the fluorescence excited by the beam during each amplification cycle: At the temperature of the end of extension phase (F_{E} at T_{E}: 70° C.), before the melting temperature of the amplified SV40 PCR product (F_{B1} at T_{B1}: 76.3° C.), beginning of the melting temperature of the amplified SV40 PCR product (F_{MS1} at T_{MS1}: 77.5° C.), and at the end of melting temperature of the amplified SV40 PCR product (F_{Me1} at T_{ME1}: 79.5° C.).

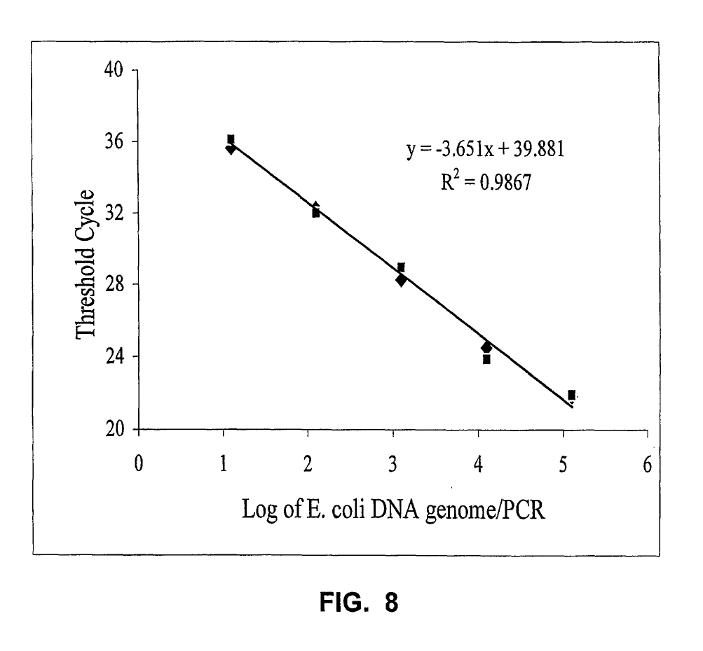

Determine the change in slope from a baseline slope (S_{B1}), defined by the negative value of (F_{B1} minus F_{E}) divided by (T_{B1} minus T_{E}), to amplicon SV40 melting phase slope (S_{M1}), defined by the negative value of (F_{ME1} minus F_{MS1}) divided by (T_{ME1} minus T_{MS1}). See FIG. 8.

Record the thermal cycle at which the first appearance of a positive change in slope occurs, where (S_{M1} minus S_{B1}) is greater than zero. Repeat the above steps 38 times to determine a range of concentrations from 2.75E+4 to 3.44E+6 copy/PCR to provide the standard curve.

Quantify the starting concentration of SV40 in SV40 DNA concentration in unknown sample. Then, compare the resultant thermal cycle number with the standard curve to determine the starting SV40 DNA concentration in unknown sample.

Results

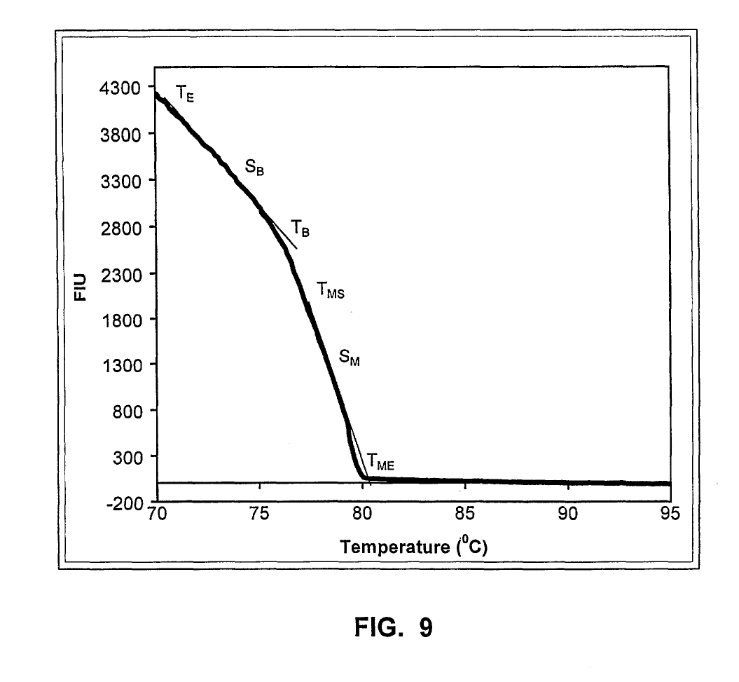

See FIG. 8 Melting profile of SV40 Amplicon, and FIG. 9 SV40 Standard Calibration Curve.

### EXAMPLE 5

Multiplex Q-PCR Assay

This Example demonstrates how the two previously described assays may be combined in a single reaction to test for both targets. The procedure is the same as for Example IV but in this study the samples include template DNA for E. coli O157:H7 and SV40 thus amplicons for both targets will be produced and measured.

Material And Methods

Cell Culture E. coli O157:H7

Each process was the same as in Example III to prepare fresh culture and dilution. The cell counts of target E. coli O157:H7 in the final were approximately 10⁸, 10⁷, 10⁶, 10⁵, 10⁴, and 0 CFU/ml respectively. The cell lysis process was the same as in Example III.

SV40 viral DNA spiked into E. coli O157:H7 cell lysate.

The same SV40 DNA as in Example IV was used in this study. A serial dilution of SV40 from 50 pg/ul into 20, 10, 4, 0.8, 0.16 and 0 pg/50 ul were spiked into E. coli O157:H7 at level of 0, 10⁴, 10⁵, 10⁶, 10⁷, and 10⁸ CFU/ml respectively. Finally, all samples were subject to PCR amplification followed by fluorescence detection in PE/ABI PRISM 7700 Sequence Detection System to determine the quantity of PCR product from different levels of E. coli O157:H7 for each PCR cycle. The final E. coli O157:H7 and SV40 DNA concentration per PCR reaction were set at 1.25E+5 E. coli genome/0 SV40, 1.25 E+4 E. coli genome/2.75E+4 copy of SV40, 1.25E+3 E. coli genome /1.38E+5 copy SV40, 1.25 E+2 E. coli genome/6.88E+5 copy SV40, 1.25E+1 E. coli genome/1.72E+6 copy SV40, and unspilced E. coli O157:H7 genome/3.44E+6 copy SV40 DNA respectively.

PCR Reagents

The PCR reagents consisted of BAX® for Screening/E. coli 0157:H7 tablets (Qualicon, Inc.) that contained proven effective concentrations of 106 mM of dATP, dCTP, dGTP, dTTP, 72 nM of primer 5′(TAC CTG AGG CAG TAG CGA TAA TGA GC)3′.33-26-rc1012; 72 nM of primer 5′(ATG CAG ACC CGC TOG AGT TTG AGA AA)3′.33-26-538 and 1.5 units of Taq™ polymerase. An additional 200 nM of SV40 primer P-4158 5′(TTA AAA AGC TAA AGG TAC ACA ATT TTT GAG CA)-3′ and 200 nM of primer rc-4289 5′(AAA AGC TGC ACT GCT ATA CAA GAA AAT TAT GG)-3′ was added to each PCR reaction. Lot 9020 tablets were used for the study. This is the same PCR reagent composition used in Example IV.

Quantitative PCR Process

Duplicate aliquots of 50 μL of each lysed sample spiked with SV40 DNA were removed and transferred into a PCR tube containing one BAX® for Screening/E. coli O157:H7 tablet, then amplified in a PE/ABI PRISM 7700 Sequence Detection System. The reaction proceeded via an initial holding period of 2 min at 94° C., followed by 38 cycles of 94° C./8 seconds and 70° C./2.53 minutes, then, (T_{E}) 70° C./7 seconds, (T_{B1}) 73.5° C. /7 sec, and (T_{MS1}) 77.5° C./7 seconds, (T_{ME1}) 78.9° C./7, (T_{B2}) 83° C./7 seconds, and (T_{ME2}) 89.4° C./7 seconds. We collected fluorescence signal from the last 6 events (F_{E} at 70° C./7 sec, F_{B1} at 73.5° C./7 see, F_{MS1} at 77.5° C./7 sec, F_{ME1} at 78.9° C./7 sec, F_{B2}/F_{MS2} and F_{ME2} at 89.4° C./7 sec) from each and every cycle. In this case, the F_{B2} was the same as F_{MS2}. After the 38 cycles were complete, the PCR tubes were held at 25° C. until analysis.

PCR Product Analysis

Establishment of the threshold cycle (C_{T}) value for distinguishing samples containing various levels of SV40 from those not containing target cells was based on the first appearance of a positive change of the SV40 amplicon melting phase slope (S_{M1}) from the baseline slope (S_{B1}) and a positive change of the E. coli O157:H7 amplicon melting phase slope (S_{M2}) from the baseline slope (S_{B2}).

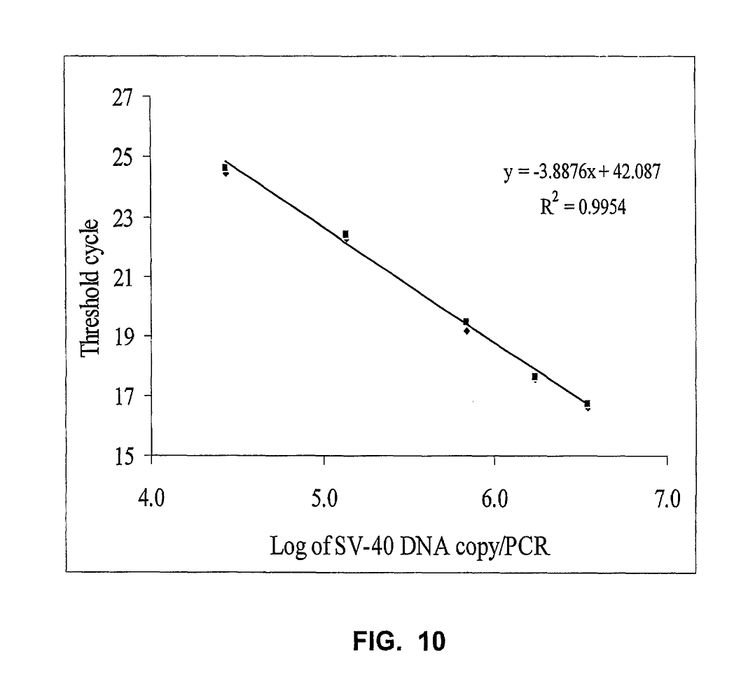

Separately, determine the change in slope for the SV40 amplicon from the baseline slope (S_{B1}), defined by the negative value of (F_{B1} minus F_{E}) divided by (T_{B1} minus T_{E}), to a melting phase slope (SM1), defined by the negative value of (F_{MS1} minus F_{ME1}) divided by (T_{MS1} minus T_{ME1}). See FIG. 10.

Determine the change in slope for the E. coli O157:H7 amplicon from the baseline slope (S_{B2}), defined by the negative value of (F_{B2} minus F_{ME1}) divided by (T_{B2} minus T_{ME1}), to amplicon melting phase slope (S_{M2}), defined by the negative value of (F_{ME2} minus F_{MS2}) divided by (T_{ME2} minus T_{MS2}). See FIG. 10.

Separately record the thermal cycle at which the first appearance of a positive change in slope for the SV40 amplicon occurs, where (S_{M1} minus S_{B1}) is greater than zero. Repeat 38 times of steps 1) through 3) for a range of concentrations of 2.75E+4 copy to 3.44E+6 copy SV40 DNA to provide the standard curve.

Quantify the starting concentration of SV40 in an unknown sample by running lysate in PCR process described above. Then, compare the resultant thermal cycle number with the standard curve to determine the starting SV40copy number in unknown sample.

Record the thermal cycle at which the first appearance of a positive change in slope for the E. coli O157:H7 amplicon occurs, where (S_{M2} minus S_{B2}) is greater than zero.

Repeat the above steps 38 times for a range of concentrations of 1.25E+1 to 1.25 E+5 E. coli O157:H7 genome/PCR to provide the E. coli O157:H7 standard curve.

Quantify the starting concentration of E. coli O157:H7 in an unknown sample by running lysate in PCR process described above. Then, compare the resultant thermal cycle number with the standard curve to determine the starting E. coli O157:H7 copy number in unknown sample.

Results

See FIG. 10 Melting profile of SV40/E. coil O157:H7 amplicon, and FIG. 11. SV40/E. coli O157:H7 Standard Calibration Curve.

### EXAMPLE 6

Single Target Quantitative PCR Assay: Additional Threshold Determinations

The previous Examples I to IV compare the change in slope of the fluorescence value of a specific amplicon to that of the baseline of the intercalating dye. The threshold cycle is defined as the cycle in which the first positive change in the amplicon slope, with respect to the baseline slope, is detected. There are other approaches for determining the threshold for the first appearance of a specific amplicon in a PCR reaction. One can assume there is no detectable production of target amplicon in the first ten PCR cycles. Therefore, a threshold fluorescence value can be calculated by averaging the fluorescence values of the first ten cycles and adding to that fifteen times the standard deviation of this fluorescence value. There are several disadvantages for this method. First, it is less specific for a target DNA since many amplicons have similar starting melting temperature. It can only apply to single target PCR quantification. It is less reproducible than the method employed in Examples 1-4, since it is affected by instrument to instrument variability, and well-to-well variability in PCR performance. It is also depends on fluorescence dye concentration in the assay which will shift the baseline signal.

An example using the CaMV 35S viral promoter will illustrate the method. A specific amplicon fluorescence signal was measured before melting and after total denaturation for every cycle. There should be no detectable amplified product fluorescence signal during the early PCR cycles. The threshold is determined as the average change in fluorescence value of the first ten cycles plus fifteen times the standard deviation of the ten fluorescence values (FE₋₁₀^{ave}+15XSD). The threshold cycle is defined as the cycle wherein the fluorescence of the sample exceeds FE₋₁₀^{ave}+15XSD. Quantification of unknown concentrations of target nucleic acid in the starting material can be extrapolated by comparing their threshold cycle (Ct) to a standard curve of Ct's generated from controls of known concentration.

Material and Methods

The same as in Example I—CaMV 35S viral DNA with known concentrations.

PCR Reagent and Process

The same as in Example I.

Quantitative PCR Assay

Pipette 25 μl of each the five levels of DNA standards (triplicate) and the unknown sample extract into a PCR well (triplicate) which content one reagent tablet. To 25 μl of each sample of extracted DNA, add 25 μl of the CAMV/buffer mixture.

Place the sample tubes into a cooling block (Qualicon, Inc.) and vortex the PCR tubes to mix the sample, reagent, and tablet. Place the rack of PCR tubes into PE/ABI 5700 Sequence Detection System (Perkin-Elmer)

PCR Parameter Set up

| | |
|---|---|
| Stage I: | 94° C. for 3 minutes |
| Stage II: | Run 40 cycles with: |
| | 94° C. for 20 seconds |
| | 70° C. for 40 seconds |
| | 72° C. for 1 minute |
| | 82° C. for 12 seconds |
| Stage III: | 72° C. for 3 minutes |

Collect the fluorescence signal from each cycle of stage II at (T_{MS}) 82° C., (T_{MT}) 94° C., in order to quantify the copy number of CAMV DNA sequences.

T_{MT}: Temperature in a PCR reaction of total melting temperature for All ds-DNA.

T_{MS}: Temperature in a PCR reaction of the beginning of the melting temperature for amplicon.

Data Process and Analysis

Measure the fluorescence excited by the beam during each amplification cycle:

At the temperature of the amplified 35S CaMV PCR product beginning to melt (F_{MS} at T_{MS}: 82° C.) and

After the total melting temperature of the amplified 35S CaMV amplicon (F_{M} at T_{MT}: 94° C.).

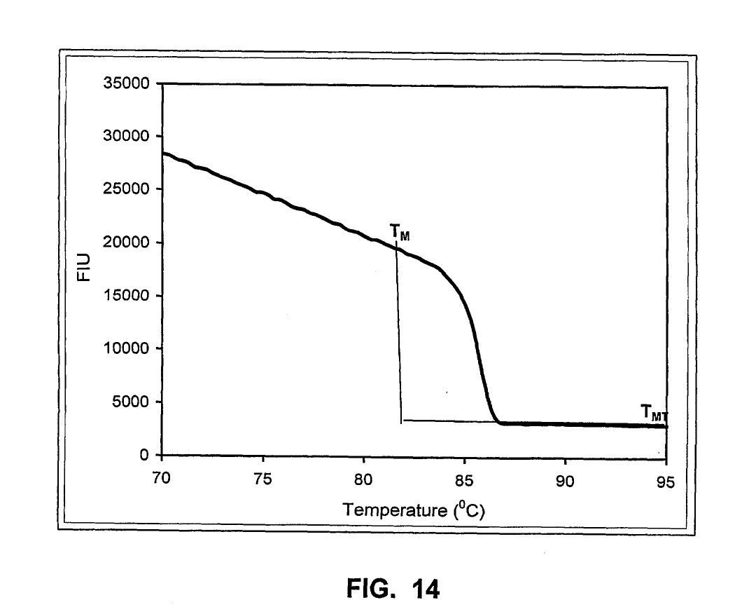

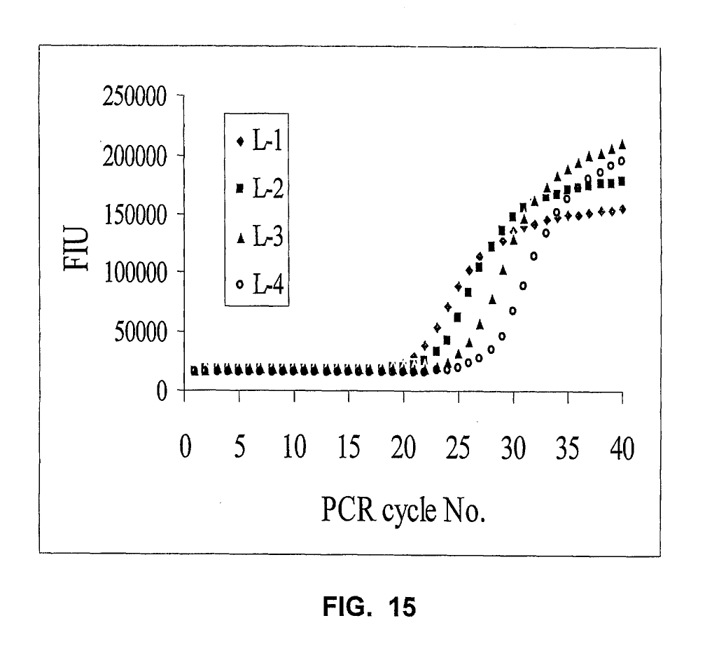

Determine the change of fluorescence signal from F_{MT} to F_{MS} of each PCR cycle, by subtract F_{MT} from F_{MS}. See FIG. 14. Record the thermal cycle at which the first appearance of a positive change, where the value is greater than threshold value. Repeat the steps above 40 times to determine a range of concentrations from 35 to 4375 copy of 35S CaMV genome/PCR to provide the standard curve. See FIG. 15.

Quantify the starting concentration of CaMV 35S promoter DNA in an unknown sample by running the same DNA extraction and PCR process described above. Then, compare the resultant thermal cycle number with the standard curve to determine the starting concentration in unknown sample.

Results

See FIG. 14 Melting Profile of CaMV Amplicon and signal determination, FIG. 15 Real-time Quantitative PCR for CaMV.

What is claimed is:

1. A method for detecting in real time the amount of a target nucleic acid molecule in a sample, wherein the melting of the target nucleic acid molecule starts at a temperature T_{MS} and completes at a temperature T_{ME}, the method comprising:
A. Establishing a standard curve by:
i) PCR-amplifying, in the presence of a suitable fluorescent dye, the target nucleic acid molecule, with a known starting concentration (C) through cycles of denaturing, annealing, and chain extension, wherein the fluorescence is increased when the dye is combined with a double-stranded nucleic acid molecule, wherein the chain extension occurs at a chain extension temperature T_{E};
ii) measuring the fluorescence (F) during each amplification cycle at the temperature immediately before the temperature starts to increase from T_{E} (F_{E} at T_{E}), at any temperature point (T_{B}) in between T_{E} and T_{MS} (S_{B} at T_{B}), at T_{MS} (F_{MS} at T_{MS}) and at T_{ME} (F_{ME} at T_{ME});
iii) calculating a baseline slope (S_{B}), defined by negative (F_{B} minus F_{E}), divided by (T_{B} minus T_{E}), and an amplicon melting phase slope (S_{M}), defined by negative (F_{ME} minus F_{MS}) divided by (T_{ME} minus T_{MS});
iv) recording the number of PCR cycles (N) required for the quantity (S_{M} minus S_{B}) to first become greater than zero;
v) repeating steps i) through v) for a suitable range of concentrations of interest; and
vi) plotting C against C_{T} to obtain a standard curve for the target nucleic acid sequence; and
B. Repeating steps (A)(i) through (A)(v) for a sample containing an unknown concentration of the target nucleic acid molecule, to obtain an C_{T} value for the sample, and determining the target nucleic acid molecule concentration via the standard curve.

2. The method of claim 1 wherein the sample contains a first target nucleic acid molecule and a second target nucleic acid molecule, wherein the melting of the first target nucleic acid molecule starts at a temperature T_{MS1} and completes at a temperature T_{ME1}, the melting of the second target nucleic acid molecule starts at a temperature T_{MS2} and completes at a temperature T_{ME2}, and wherein T_{MS2} is greater than T_{ME1}, the method comprising:
A. Establishing a standard curve for each of the target nucleic acid molecule by:
i) simultaneously PCR-amplifying, in the presence of a suitable fluorescent dye, the target nucleic acid molecules with a known starting concentration (C₁ and C₂) through cycles of denaturing, annealing, and chain extension, wherein the fluorescence is increased when the dye is combined with a double-stranded nucleic acid molecule, wherein the chain extension occurs at a chain extension temperature T_{E};
ii) measuring the fluorescence (F) during each amplification cycle at the temperature immediately before the temperature starts to increase from T_{E} (F_{E} at T_{E}), at any temperature point (T_{B1}) in between T_{E} and T_{MS1} (F_{B1} at T_{B1}), at T_{MS1} (F_{MS1} at T_{MS1}), at T_{ME1} (F_{ME1} at T_{ME1}), at any time point (T_{B2}) in between T_{ME1} and T_{MS2} (F_{B2} at T_{B2}), at T_{MS2} (F_{MS2} at T_{MS2}), at T_{ME2} (F_{ME2} at T_{ME2});
iii) calculating a baseline slope for the first target molecule (S_{B1}), defined by negative (F_{B1} minus F_{E}), divided by (F_{B1} minus T_{E}), and a first amplicon melting phase slope for the first molecule (S_{M1}), defined by negative (F_{ME1} minus F_{MS1}) divided by (T_{ME1} minus T_{MS1}); and calculating a baseline slope for the second target molecule (S_{B2}), defined by negative (F_{B2} minus F_{ME1}), divided by (T_{B2} minus T_{ME1}), and a melting phase slope for the first molecule (S_{M2}), defined by negative (F_{ME2} minus F_{MS2}) divided by (T_{ME2} minus T_{MS2});
iv) recording the number of PCR cycles (N₁) required for the quantity (S_{M1} minus S_{B1}) to first become greater than zero; and recording the number of PCR cycles (N₂) required for the quantity (S_{M2} minus S_{B2}) to first become greater than zero;
v) repeating steps i) through v) for a suitable range of concentrations of interest for each of the two target molecules; and
vi) plotting C₁ against N₁ to obtain a standard curve for the first target molecule; and plotting C₂ against N₂ to obtain a standard curve for the second target molecule; and
B. Repeating steps (A)(i) through (A)(v) for a sample containing an unknown concentration of the first and second target nucleic acid molecules, to obtain an N₁ value and an N₂ value for the sample, and determining the target nucleic acid molecule concentrations via the standard curve.

3. The method of claim 1 wherein the sample contains a first target nucleic acid molecule, a second target nucleic acid molecule and a third target nucleic acid molecule, wherein the melting of the first target nucleic acid molecule starts at a temperature T_{MS1} and completes at a temperature T_{ME1}, the melting of the second target nucleic acid molecule starts at a temperature T_{MS2} and completes at a temperature T_{ME2}, the melting of the third target nucleic acid molecule starts at a temperature T_{MS3} and completes at a temperature T_{ME3}, and wherein T_{MS3} is greater than T_{ME2}, the method comprising:
A. Establishing a standard curve for each of the target nucleic acid molecule according to the method of claim 1;
B. Simultaneously PCR amplifying a sample containing an unknown concentration of the target nucleic acid molecules, to obtain an N₁, N₂ and N₃ value for the sample, and determining the target nucleic acid molecule concentrations via the standard curve.

4. The method of claim 1 wherein the sample contains n target nucleic acid molecules, wherein n is an integer greater than three, wherein the melting of the first target nucleic acid molecules starts at a temperature T_{MS1} and completes at a temperature T_{ME1}, the melting of the second target nucleic acid molecule starts at a temperature T_{MS2} and completes at a temperature T_{ME2}, the melting of the (n−1)^{th} target nucleic acid molecule starts at a temperature T_{MS(n-1)} and completes at a temperature T_{ME(n-1)}, the melting of the n^{th} target nucleic acid molecule starts at a temperature T_{MSn} and completes at a temperature T_{MEn}, and wherein T_{MSn} is greater than T_{ME(n-1)}, the method comprising:
A. Establishing a standard curve for each of the target nucleic acid molecule according to the method of claim 1;
B. Simultaneously PCR amplifying a sample containing an unknown concentration of the target nucleic acid molecules, to obtain an N₁, N₂ . . . and Nₙ value for the sample, and determining the target nucleic acid molecule concentrations via the standard curve.

5. The method of claim 2, wherein the first and the second target nucleic acid molecules reside on the same genome of an organisms, and wherein the copy number per genome for the first target nucleic acid molecule is known, whereby the copy number per genome for the second target nucleic acid molecule is determined.

6. The method of claim 1, wherein the target nucleic acid molecule is from a pathogenic organisms.

7. The method of claim 2, wherein the first target nucleic acid is an invertase gene, an aldolase gene or a lectin gene, and wherein the second target nucleic acid is selected from the group consisting of the 35S CaMV promoter, a Cry9C gene, an GA21 gene, an EPSPS (5-enolpyruvylshikimate-3-phosphate synthase gene, a PEPC promoter; an hsp70 promoter of Cry1A(b) gene, a Cry1A(b) gene; an NOS gene, and the actin promoter gene.

8. The method of claim 1 wherein the target nucleic acid molecule is selected from the group consisting of SEQ ID NO: 11, SEQ ID NO: 12, SEQ ID NO: 9, and SEQ ID NO: 10.

9. The method of claim 1, wherein the target nucleic acid molecule is a nucleic acid fragment is part of a transgene contained in a genetically modified organism. The method of claim 9 wherein the target nucleic molecule comprises a promoter for the transgene.

10. The method of claim 10 wherein the promoter is the 35S promoter of Cauliflower Mosaic Virus.
